(19) United States
(12) Patent Application Publication
(10) Pub. No.: US 2021/0046125 A1
(43) Pub. Date: Feb. 18, 2021

(54) DECELLULARIZED TISSUES, HYDROGELS THEREOF, AND USES THEREOF

(71) Applicant: UNIVERSITY OF FLORIDA RESEARCH FOUNDATION, Gainesville, FL (US)

(72) Inventors: Nikunj Agrawal, Gainesville, FL (US); Jonathan Griffin, Gainesville, FL (US); Christine E. Schmidt, Gainesville, FL (US); Michaela W. McCrary, Gainesville, FL (US); Deanna Bousalis, Gainesville, FL (US); Young Hye Song, Gainesville, FL (US)

(21) Appl. No.: 16/968,941

(22) PCT Filed: Feb. 21, 2019
(86) PCT No.: PCT/US2019/019001

§ 371 Date: Aug. 11, 2020

Related U.S. Application Data
(60) Provisional application No. 62/655,474, filed on Apr. 10, 2018.
(60) Provisional application No. 62/633,155, filed on Feb. 21, 2018.

(51) Int. Cl. A61K 35/34; A61L 27/52; A61L 27/36; A61L 27/54

(52) U.S. Cl. CPC A61K 35/34; A61L 27/54; A61L 27/3633; A61L 27/52

(57) ABSTRACT

Described herein are methods of producing decellularized tissue hydrogels. In some aspects, the decellularized tissue hydrogels can contain one or more extracellular matrix proteins. Also described herein are methods of making the decellularized tissue hydrogels. Also described herein are methods of using the decellularized tissue hydrogels. In some aspects, the decellularized tissue hydrogels or a pre-gel solution can be administered to a subject.

### CROSS-REFERENCE TO RELATED APPLICATIONS

This application claims the benefit of and priority to co-pending U.S. Provisional Patent Application No. 62/633,155 filed on Feb. 21, 2018, entitled “TISSUE DECELLULARIZATION METHODS,” the contents of which is incorporated by reference herein in its entirety.

This application also claims the benefit of and priority to co-pending U.S. Provisional Patent Application No. 62/655,474 filed on Apr. 10, 2018, entitled “TISSUE DECELLULARIZATION METHODS,” the contents of which is incorporated by reference herein in its entirety.

### STATEMENT REGARDING FEDERALLY SPONSORED RESEARCH

This invention was made with Government support under grant no. P0004879 awarded by the National Science Foundation. The Government has certain rights in this invention.

### BACKGROUND

Tissue scaffolds play key roles in tissue engineering, repair of soft tissues, and can serve as drug delivery platforms. As such, there exists a need to generate tissue scaffolds.

### SUMMARY

Described herein are embodiments of a decellularized tissue hydrogel that can be composed of decellularized tissue, wherein the decellularized tissue can contain native extracellular matrix proteins, wherein the decellularized tissue is cross-linked to form the hydrogel. The decellularized tissue hydrogel can further include a therapeutic agent. The therapeutic agent can be a population of cells, a polynucleotide, an amino acid, a peptide, a polypeptide, an antibody, an aptamer, a ribozyme, a guide sequences for a ribozyme that inhibit translation or transcription of essential tumor proteins and genes, a hormone, an immunomodulator, an antipyretic, an anxiolytic, an antipsychotic, an analgesic, an antispasmodic, an anti-inflammatory agent, an anti-histamine, an anti-infective, a chemotherapeutic, or a combination thereof. The decellularized tissue can be decellularized brain, spinal cord, heart, lung, liver, muscle, cartilage, tendons, ligament, menisci, stomach, intestine, pancreas, kidney, blood vessel, bone, or cornea tissue. The decellularized tissue hydrogel can be formed by the method that can include the steps of:

(a) obtaining an amount of tissue from a donor;

(b) inducing widespread apoptosis in the tissue;

(c) removing apoptotic bodies using a mild hyper or hypo tonic buffered solution;

(d) removing DNA by exposing the tissue from (c) to DNase;

(e) enzymatically digesting the tissue from (d)

(f ) neutralizing the tissue from (e) to form a pre-gel solution; and

(g) incubating the pre-gel solution at about 37° C. for an amount of time to form the decellularized tissue hydrogel. In some embodiments, step (b) can be performed by incubating the tissue in (a) to camptothecin. In some embodiments, step (e) can be performed by incubating the tissue from (d) in a solution including pepsin. In some embodiments, step (g) can be performed in vitro. In some embodiments, step (g) can be performed in vivo. In some embodiments, the method can further include the step of lyophilizing the tissue from step (d) before step (e),

In some embodiments, the decellularized tissue hydrogel can be formed by a method that can include the steps of:

(a) obtaining an amount of tissue from a donor;

(b) incubating the tissue from (a) in a solution containing sodium deoxycholate;

(c) removing DNA from the tissue from (b) by exposing the tissue from (b) to DNase;

(d) washing the tissue from (c) in PBS;

(e) enzymatically digesting the tissue from (d)

(f) neutralizing the tissue from (e) to form a pre-gel solution; and

(g) incubating the pre-gel solution at about 37° C. for an amount of time to form the decellularized tissue hydrogel. In some embodiments step (b) can be performed by incubating the tissue in about a 3% solution (w/w), (w/v), or (v/v) of sodium deoxycholate. In some embodiments, step (e) can be performed by incubating the tissue from (d) in a solution comprising pepsin. In some embodiments, step (g) can be performed in vitro. In some embodiments, step (g) can be performed in vivo. In some embodiments, the method can further include the step of lyophilizing the tissue from step (d) before step (e).

Also described herein are methods that can include the step of administering a decellularized tissue described herein a subject. The method can further include the step of co-administering a population of cells with the decellularized tissue hydrogel as described herein. In some embodiments, the subject has a peripheral nerve injury, spinal cord injury, spinal cord disease, amputation, has degradation of a component of the spine, osteoarthritis, lung trauma, volumetric muscle loss, or liver cirrhosis.

Also described herein are embodiments of a pre-gel solution that can include decellularized tissue, wherein the decellularized tissue can contain native extracellular matrix proteins, and wherein the decellularized tissue is capable of forming a hydrogel at about 37° C. In some embodiments, the pre-gel solution can be at about 4° C. to about 25° C. The pre-gel solution can further include a therapeutic agent. The therapeutic agent can be a population of cells, a polynucleotide, an amino acid, a peptide, a polypeptide, an antibody, an aptamer, a ribozyme, a guide sequences for a ribozyme that inhibit translation or transcription of essential tumor proteins and genes, a hormone, an immunomodulator, an antipyretic, an anxiolytic, an antipsychotic, an analgesic, an antispasmodic, an anti-inflammatory agent, an anti-histamine, an anti-infective, a chemotherapeutic, or a combination thereof. The decellularized tissue can be decellularized brain, spinal cord, heart, lung, liver, muscle, cartilage, tendons, ligament, menisci, stomach, intestine, pancreas, kidney, blood vessel, bone, or cornea tissue.

In some embodiments, the pre-gel solution is formed by a method that can include at least the steps of:

(a) obtaining an amount of tissue from a donor;

(b) inducing widespread apoptosis in the tissue;

(c) removing apoptotic bodies using a mild hyper or hypo tonic buffered solution;

(d) removing DNA by exposing the tissue from (c) to DNase;

(e) enzymatically digesting the tissue from (d); and

(f) neutralizing the tissue from (e) to form a pre-gel solution.

In some embodiment, step (b) can be performed by incubating the tissue in (a) to camptothecin. In some embodiments, step (e) can be performed by incubating the tissue from (d) in a solution comprising pepsin. The method can further include the step of lyophilizing the tissue from step (d) before step (e).

In some embodiments, the pre-gel solution can be formed by a method that can include at least the steps of

(a) obtaining an amount of tissue from a donor;

(b) incubating the tissue from (a) in a solution containing sodium deoxycholate;

(c) removing DNA from the tissue from (b) by exposing the tissue from (b) to DNase;

(d) washing the tissue from (c) in PBS;

(e) enzymatically digesting the tissue from (d); and

(f) neutralizing the tissue from (e) to form a pre-gel solution.

In some embodiments, step (b) can be performed by incubating the tissue in about a 3% solution (w/w), (w/v), or (v/v) of sodium deoxycholate. Step (e) can be performed by incubating the tissue from (d) in a solution comprising pepsin, In some embodiments, the method can further include the step of lyophilizing the tissue from step (d) before step (e).

Also described herein are embodiments of a method that can include the step of injecting an amount of a pre-gel solution as described herein into a subject. In some embodiments, the method can include the step of co-administering a population of cells with the pre-gel solution in any one of claims 20-32. In some embodiments, the subject can have a peripheral nerve injury, spinal cord injury, spinal cord disease, amputation, has degradation of a component of the spine, osteoarthritis, lung trauma, volumetric muscle loss, or liver cirrhosis.

### BRIEF DESCRIPTION OF THE DRAWINGS

Further aspects of the present disclosure will be readily appreciated upon review of the detailed description of its various embodiments, described below, when taken in conjunction with the accompanying drawings.

Further aspects of the present disclosure will be readily appreciated upon review of the detailed description of its various embodiments, described below, when taken in conjunction with the accompanying drawings.

FIG. 1 shows one embodiment of a method for decellularization of a tissue sample.

FIGS. 2A-2F show fluorescence micrographs of peripheral nerve with and without apoptosis induction followed by washing with various regimens.

FIG. 3 shows a graph demonstrating DNA content of peripheral nerve under various treatment regimens.

FIG. 4 shows a table demonstrating the quantification of DNA content shown in FIG. 3.

FIGS. 5A-5C demonstrate fluorescence micrographs of nerve tissue labeled for cell or basal lamina markers. For FIGS. 5A and 5B, the images are fresh nerve (top half) and apoptosis decellularized nerve (bottom half). FIG. 5A was labeled for neurons (neurofilament, green), Schwann cells (S100, red), and nuclei (DAPI, blue). FIG. 5B shows cross-sections of the basal lamina stained for laminin (red). FIG. 5C shows a representative basal lamina staining in nerve tissue decellularized according to a conventional detergent-based method.

FIGS. 6A-6D demonstrate fluorescence micrographs of nucleus pulposus stained with a nuclear stain (blue) and a chondroitin sulfate proteoglycan (CSPG) antibody (red).

FIGS. 7A-7B demonstrate images of a carefully dissected motion segment of the spine and an extracted nucleus pulposus.

FIG. 8 shows a graph demonstrating tibialis anterior muscle weight changes over time in rats receiving detergent decellularized nerve graft, apoptosis decellularized nerve graft, or an isograft of fresh nerve. This figure demonstrates that the apoptosis decellularization method was not statistically different from isograft after 8 weeks, suggesting that the apoptosis decellularization method has the potential to perform similarly to the clinical gold standard and outperform traditional decellularization methods.

FIGS. 9A-9C show fluorescence images demonstrating that the more gentle apoptosis decellularization (FIG. 90) process can result in less tissue disruption than the harsher traditional decellularization methods which uses a water treatment to initiate decellularization (FIG. 9B), and more comparable to fresh nerve (FIG. 9A).

FIG. 10 shows a flow diagram demonstrating the general steps in forming a decellularized tissue hydrogel using apoptotic decellularization followed by enzymatic digestion and thermal gelation. After the fresh tissue is harvested, the tissue can be decellularized using chemical and apoptotic decellularization process (1.) to obtain a decellularized tissue. Enzymatic and further processing (2) can be performed to yield a solution that can undergo thermal gelation (3.) to form a tissue hydrogel (ECM hydrogel).

FIG. 11 shows a table demonstrating steps in an embodiment of an apoptosis decellularization method. This method can be followed by enzymatic digestion and thermal gelation.

FIGS. 12A-12B show results of implantation of a tissue hydrogel prepared from rat sciatic nerve injected into a spinal cord along with Schawnn cells (SC), FIG. 10A shows a confocal image showing SC transplantation using decellularized nerve hydrogels in a spinal cord section (green is SC, red is astrocytes). FIG. 10B shows transmission electron microscopy (TEM) image that can demonstrate myelination (dark outlines) along axons (circular bodies).

FIG. 13 shows a graph that can demonstrate the results of a DNA assay (Promega QuantiFlour) applied to measure residual DNA content in tissue. FIG. 13 can demonstrate that DAN is significantly reduced in apoptosis decellularized (AD) nerve tissue.

FIGS. 14A-14F shows results from histological analysis (H&E staining, FIGS. 14A and 14D) and immunoflorescent imaging (FIGS. 14B-14C and 14E-14F) in fresh nerve tissue (FIGS. 14A-14C) and apoptosis decellularized (AD) nerve tissue.

FIG. 15 shows a graph that can demonstrate cytotoxicity of Schwann cells cultured in media eluted from apoptosis decellularized (AD) nerve tissue, chemically decellularized (CD) nerve tissue, fresh nerve tissue, and fresh media. AlamarBlue reduction suggested that only AD nerve and fresh media groups did not alter metabolic activity of the cultured Schwann cells.

FIGS. 16A-16D show fluorescent microscopy images that can demonstrate subcutaneous implantation of decellularized nerves into rats allowed for analysis of an immune reaction. Macrophage (green) and nuclear (blue) staining can demonstrate that macrophage infiltration was lowest in AD nerves (FIG. 16A) as compared to CD nerves (FIG. 16B), isografts (FIG. 16C), and allografts (FIG. 16D).

FIGS. 17A-17B show the effect of different thermal gelation conditions. Thermal gelation was performed via enzymatic digestion of AD nerves followed by incubation of a precursor solution at about 37° C. for up to about 60 minutes (data shown here is at 30 minute incubation and 45 minute incubation. Gelation was confirmed qualitatively by placing gels in 1X PBS. Precursor solutions containing AD nerve tissue incubated for 30, 45 or 60 minutes produced gels that maintained their shape indicating thermal gelation in these cases was achieved.

FIG. 18 shows a graph that can demonstrate the turbidity gelation kinetics of the tissue hydrogels formed by measuring the absorbance (405 nm) of one sample over time while incubating at about 37 over time as gelation occurs. The graph in FIG. 18 can suggest that full gelation can occur shortly after about 30 minute incubation.

FIGS. 19A-19B show tissue hydrogels formed from chemically decellularized tissue with initial tissue concentrations (FIG. 19A) 7.5 mg/mL, (FIG. 10B) 10 mg/mL, and following 15 minutes in 37° C.

FIG. 20 shows a graph that can demonstrate the normalized absorbance of hydrogels generated from chemically decellularized nerve which depicts the formation of the hydrogel when incubated at 37° C.

FIG. 21 shows images of the hydrogels formed from chemically decellularized lung tissue and results from integrity tests (submersion in PBS) are shown in FIG. 21.

FIG. 22 shows a graph that can demonstrate the gelation kinetics from chemically decellularized lung tissue. The absorbance at 405 nm was measured over time during gelation incubation for 60 minutes.

FIGS. 23A-23B shows results from confocal collagen reflectance from chemically decellularized lung tissue

FIG. 24 shows an image of a lung tissue hydrogel formed as described above, 10 mg/mL lung tissue (ECM) hydrogel is shown here.

FIG. 25 shows a graph demonstrating the gelation kinetics of the lung tissue hydrogel shown in FIG. 24.

FIG. 26 shows an image of a lung tissue hydrogel formed using 7.5 mg/mL of tissue for gelation as described above.

FIG. 27 shows a graph of the gelation kinetics of the 7.5 mg/mL tissue hydrogel.

FIG. 28 shows a flow diagram demonstrating general steps in a chemical decellularization method that can result in an acellular tissue product that retains one or more extracellular matrix proteins.

FIG. 29 shows a flow diagram demonstrating general steps to form a decellularized tissue hydrogel from the decellularized tissues made from the apoptosis or chemical decellularization methods described herein.

FIG. 30 shows a graph that can demonstrate rheological properties of an iPN. This graph can demonstrate the shear modulus for low and high concentration iPN (7.5 and 10 mg/ml). These are shown against pure collagen-I hydrogels at similar concentrations for reference. iPN hydrogels derived using the sodium deoxycholate decellularization method.

FIG. 31 shows a panel of confocal reflectance images of low and high iPN concentration hydrogels (7.5 and 10 mg/ml) and these are shown against pure collagen-I hydrogels at similar concentrations for reference. iPN hydrogels derived were from the new sodium deoxycholate decellularization method.

FIG. 32 shows a graph that can demonstrate the shear modulus for low and high concentration iPN (7.5 and 10 mg/ml), These are shown against pure collagen-I hydrogels at similar concentrations for reference, iPN hydrogels derived using the sodium deoxycholate decellularization method.

FIGS. 33A and 33B show graphs that can demonstrate =in vitro degradation rates shown as cumulative weight loss (%) over the course of 30 days for low (7.5mg/ml) and high (10mg/ml) iPN hydrogels derived from the sodium deoxycholate decellularization method. These are shown against pure collagen-I hydrogels at similar concentrations for reference.

FIGS. 34A-34B show graphs that can demonstrate Pseudo-drug (FITC-DEXTRAN) release profiles for low and high concentration iPN (7.5 and 10 mg/ml) generated from the new sodium deoxycholate decellularization method. These are shown against pure collagen-I hydrogels at similar concentrations for reference.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g, the phrase “x to y” includes the range from ‘x’ to ‘y’ as well as the range greater than ‘x’ and less than ‘y’. The range can also be expressed as an upper limit, e.g. ‘about x, y. z. or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value.

Similarly, when values are expressed as approximations, by use of the antecedent “about,” it will be understood that the particular value forms a further aspect, For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0,5% to about 3.2%, and about 0.5% to about 4.4%, and other possible sub-ranges) within the indicated range.

As used in the specification and the appended claims, the singular forms “a,” “an,” and “the” include plural referents unless the context clearly dictates otherwise.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +1-10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible unless the context clearly dictates otherwise.

Definitions

As used herein, “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes but is not limited to, subcutaneous, intravenous, intramuscular, intra-articular, intra-synovial, intrasternal, intrathecal, intrahepatic, intralesional, and intracranial injections or infusion techniques.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “control” is an alternative subject or sample used in an experiment for comparison purposes and included to minimize or distinguish the effect of variables other than an independent variable.

As used herein, “positive control” refers to a “control” that is designed to produce the desired result, provided that all reagents are functioning properly and that the experiment is properly conducted.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “autologous” refers to being derived from the same subject hat is the recipient.

As used herein, “allograft” refers to a graft that is derived from one member of a species and grafted in a genetically dissimilar member of the same species.

As used herein “xenograft” or “xenogeneic” refers to a substance or graft that is derived from one member of a species and grafted or used in a member of a different species.

As used herein, “autograft” refers to a graft that is derived from a subject and grafted into the same subject from which the graft was derived.

As used herein, “allogeneic” refers to involving, derived from, or being individuals of the same species that are sufficiently genetically different so as to interact with one another antigenically.

As used herein, “donor” refers to a subject from which cells or tissues are derived.

As used herein, “hypertonic solution” refers to a solution that has a greater concentration of solutes than the concentration of solutes inside of a cell.

As used herein, “hypotonic solution” refers to a solution that has a concentration of solutes that is less than the concentration of solutes within a cell.

As used herein, “analog” refers to another compound, composition, or other substance of matter that is considered comparable to the reference compound, composition, or substance of matter and includes structural analogs and functional analogs.

### Discussion

Decellularization technology offers the potential to attain tissue-specific scaffolds that guide tissue regeneration following injury and/or disease. Most decellularization protocols used in research and industry contain an initial cell lysis step in a hypotonic buffer, such as water, followed by harsh chemical solutions to remove cellular remnants. These harsh conditions result in an undesirable broad dispersal of intracellular components, disruption of tissue morphology, and removal of desired tissue elements.

With that said, described herein are decellularization methods that do not require cell lysis buffers or harsh chemicals. The decellularization methods described herein can include inducing apoptosis in ex vivo tissue. The decellularization methods described herein can effectively remove cellular components without having to rely on harsh conditions. In some embodiments, the methods can effectively remove cellular components from peripheral nerve and/or nucleus pulposus. In addition to reducing time, labor, and expense of decellularization, some embodiments described herein can only need one step for inducing apoptosis and one step for washing the cells.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Apoptosis-Based Tissue Decellularization Methods

Described herein are methods of decellularization that include inducing apoptosis in an ex vivo tissue sample, where tissue wide apoptosis can be induced by exposing the tissue to an apoptosis-inducing agent or apoptosis inducing process for a time period. Induction of apoptosis in the ex vivo tissue sample can cause cell detachment from the tissue extracellular matrix, degradation of intracellular DNA, RNA and proteins, and allocation into apoptotic bodies, which can be removed using mild hypotonic and/or mild hypertonic buffered solutions.

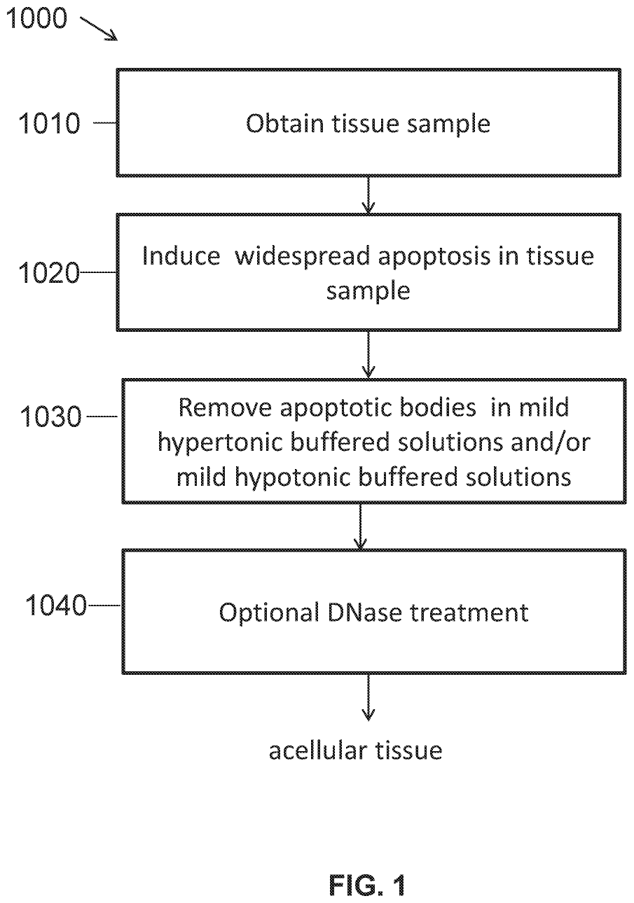

As shown in FIG. 1, the apoptotic decellularization method 1000 can begin with obtaining a tissue sample from a subject or other donor source 1010. The tissue can be autologous, xenogeneic, allogeneic, or syngeneic. In some embodiments, the tissue can be obtained from anywhere in a subject, In some embodiments, the tissue sample can be obtained from the periphery or spinal column of a subject. In some embodiments, the tissue can be a peripheral nerve and/or nucleus pulposus. In other embodiments, the tissue can be brain, spinal cord, heart, lung, liver, muscle, cartilage, tendons, ligaments, menisci, stomach, intestine, pancreas, and/or kidney. In further embodiments, the tissue is blood vessels, bone, and/or cornea. The amount of tissue can range from about 0.1 μg to 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 2.75 mg, 5 mg, 5.25 mg, 5.5 mg, 5.75 mg, 6 mg, 6.25 mg, 6.5 mg, 6.75 mg 7 mg, 7.25 mg, 7.5 mg, 7.75 mg. 8 mg, 8.25 mg, 8.5 mg, 8.75 mg, 9 mg, 9.25 mg, 9,5 mg, 9.75 mg, 10 mg, 11 mg, 12 mg, 13 mg, 14 mg, 25 mg, 16 mg, 17 mg, 18, mg, 19, mg, 20 mg, or more

After the tissue sample has been obtained, apoptosis can be induced 1020 in the ex vivo tissue sample by exposing the ex vivo tissue sample to an apoptotic agent (i.e a compound and/or composition that induces apoptosis in a cell) for a time period. In some embodiments, apoptosis can be induced by an apoptotic process (i.e. a process that can include one or more steps that induces apoptosis). In some embodiments, the apoptotic agent can be camptothecin, staurosporine, doxorubicin, and/or analogs thereof, or any other agent or process that induces apoptosis. In other embodiments, the apoptotic agent could be nitric oxide, hypoxia, pH, or hydrogen peroxide. In some embodiments where an apoptotic agent is used to induce apoptosis, the ex vivo tissue sample can be exposed to the apoptotic agent for any length of time, including but not limited to, greater than about 1 hour, about 1 hour to about 10 days, about 1 hour to about 7 days, about 1 hour to about 4 days, about 1 hour to about 2 days, or about 1 hour to about 1 day. In some embodiments, the ex vivo tissue sample can be exposed to camptothecin or analog thereof for 2 days. In some embodiments, the tissue ex vivo tissue sample can be exposed to the apoptotic agent, such as camptothecin, for about 1 day.

The ex vivo tissue sample can be exposed to a solution containing a concentration of the apoptotic agent and/or to an apoptotic process. In some embodiments, the apoptotic process does not use an apoptotic agent. In embodiments that employ an apoptotic agent, the concentration of the apoptotic agent can range from about 1 nM to about 1 mM or greater. In some embodiments, the concentration of the apoptotic agent can range from about 1 μM to about 100 μM. In some embodiments, the concentration of the apoptotic agent can be about 100. In some embodiments, the ex vivo tissue sample can be exposed to 10 μM of camptothecin or an analog thereof. In some embodiments, the ex vivo tissue sample can be exposed to 10 μM of camptothecin or an analog thereof for about 2 days. In some embodiments, the concentration of the apoptotic agent can range from about 5 μM to about 10 μM. In some embodiments, the concentration of the apoptotic agent is about 5 μM. In some embodiments, the ex vivo tissue sample can be exposed to 5 μM camptothecin or an analog thereof. In some embodiments, the ex vivo tissue sample can be exposed to 5 μM of camptothecin or an analog thereof for about 1 day.

In some embodiments, the apoptotic process includes exposure of the ex vivo tissue to one or more freeze-thaw cycles to induce apoptosis.

After the ex vivo tissue sample can be exposed to an apoptotic agent or process for a desired amount of time, the tissue sample can be washed 1030 with one or more washes with one or more buffered solutions to facilitate cell removal. The ex vivo tissue sample can be washed with one or more washes of a hypertonic buffered solution (i.e. greater than 1× buffered solution). In some embodiments the number of washes ranges from one to six, In some embodiments, the hypertonic solution can be a 1.01×-10× buffered solution. In some embodiments, the concentration can be 4× with an intermediate 2× wash. The tissue sample can be washed with one or more washes of a hypotonic buffered solution (i.e. less than 1× buffered solution). In some embodiments, the hypotonic solution can be a 0.01× up to a 0.99× buffered solution, In embodiments, the buffered solution is a saline solution at the given concentrations to result in a hypotonic or hypertonic solution. The starting saline solution can be isotonic. The starting saline solution can have a formulation of 10× which is diluted with water to attain various hypertonic washes. While not being bound to theory, it is believed that DNA removal occurs under the hypotonic wash conditions, while protein removal occurs under the hypertonic wash conditions. Some embodiments employ only hypertonic washes, Other embodiments employ only hypotonic washes. Yet further embodiments employ both hypertonic and hypotonic washes. In some embodiments, the method 1000 can also include an optional step of DNAse treatment 1040. The DNAse treatment can occur after the wash(es) previously performed 1030. In some embodiments, the optional DNAse treatment 1040 is performed after hypotonic washes 1030 were performed. In some embodiments where the optional DNAse 1040 treatment is performed, only hypertonic washes have been performed. In embodiments, the concentration of DNAse can range from 25 U/mL to 250 U/mL. The preferred concentration is 75 U/mL. The DNase can be any suitable DNase, The DNase treatment can be performed at a temperature ranging from about 4 to about 37° C. The preferred temperature is 25° C. It will be appreciated by those of ordinary skill in the art that the temperature can be dependent on the temperature that the DNase has optimal activity. The time period for the DNase treatment can range from about 30 minutes or less to 1, 2, 3, 4, 5, 6, 7, 8, 9, 10, 11, 12, 24, or 36 hours or more. The treatment time can vary based on tissue type, but in some embodiments the treatment time is 18-24 hours.

After the optional DNase treatment or prior wash(es), the resulting decellularized tissue (also referred to herein as an acellular tissue product or scaffold) can be optionally washed. The decellularized tissue can be immediately used (e.g. implanted) or can be lyophilized or otherwise stored for later processing or use. In some embodiments, the resulting decellularized tissue can undergo thermal gelation to form a hydrogel. This is discussed in greater detail elsewhere herein.

The method 1000 can produce an acellular tissue. The resulting acellular tissue can be used as a scaffold substrate for three dimensional (3D) cell culture and tissue engineering techniques. The acellular tissue can contain extracellular matrix proteins, including laminin and collagen. Further, the resulting acellular tissue can be delivered to a subject in need thereof to facilitate the in vivo generation and/or regeneration of cells and tissues within the subject. The resulting acellular tissue product can be formulated to be delivered to a subject in need thereof by any suitable method of deliver, including but not limited to, implantation or injection. The acellular tissue products can be enhanced by the addition of one or more other agents or compounds to the acellular tissue product, including without limitation other cells (e,g. stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. In some embodiments, the acellular tissue products can be co-administered with one or more other agents or compounds, including without limitation other cells (e.g. stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

In some embodiments, any of the acellular tissue scaffolds described herein can be administered to a subject in need thereof in the original form, as an injectable formulation, or as other derivatives of the acellular tissue. In some embodiments, the subject in need thereof has a peripheral nerve injury, spinal cord injury, spinal cord disease, amputation or otherwise suffers from degradation of a component of the spine (e.g intervertebral disc degeneration), In other embodiments, the subject could have osteoarthritis of the hip or knee, trauma to the lungs, volumetric muscle loss, cirrhosis of the liver, or otherwise requires partial or total organ replacement,

Chemically-Based Tissue Decellularization Methods

As discussed above, conventional harsh chemical processing of tissues used to decellularize tissue can strip the tissue completely of extracellular matrix proteins. Described herein are chemical decellularization methods that can produce an acellular tissue scaffold that retains one or more extracellular matrix proteins, including laminin, collagen (e.g. at least collagen-IV and −I).

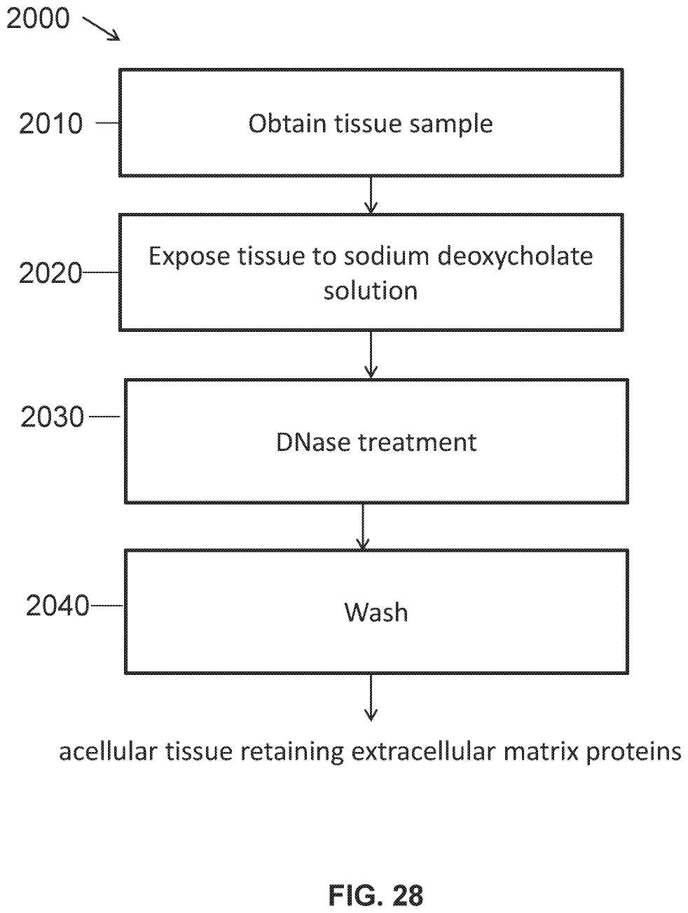

FIG. 28 shows a flow diagram demonstrating the general process for a chemical decellularization process 2000 that can generate an acellular tissue scaffold that retains extracellular matrix proteins, including collagen and laminin. Generally, an amount of tissue can be obtained 2010. The tissue sample can be obtained from a subject or other donor source 1010. The tissue can be autologous, xenogeneic, allogeneic, or syngeneic. In some embodiments, the tissue can be obtained from anywhere in a subject. In some embodiments, the tissue sample can be obtained from the periphery or spinal column of a subject. In some embodiments, the tissue can be a peripheral nerve and/or nucleus pulposus. In other embodiments, the tissue can be brain, spinal cord, heart, lung, liver, muscle, cartilage, tendons, ligaments, menisci, stomach, intestine, pancreas, and/or kidney. In further embodiments, the tissue is blood vessels, bone, and/or cornea.

The tissue can be washed after initial harvesting. In some embodiments the initial wash can be in to a solution containing sodium deoxycholate 2020. The percent sodium deoxycholate (SD) in the solution can be about 3% (w/v), In some embodiments the percent sodium deoxycholate can be about 2-5% (w/v.) The incubation in the SD solution can be about 2 hours. In some embodiments, the SD solution can contain a saline buffer and/or an amount of sulfobetaine 16. In some embodiments, the concentration of sulfobetaine-16 can be about 0.6 mM. In some embodiments, the saline buffer is a sodium/phosphate buffer. In some embodiments, the sodium/phosphate buffer can contain about 50 mM sodium. In some embodiments, the sodium/phosphate buffer can contain about 10 mM phosphate buffer. In some embodiments, the saline buffer contains about 50 mM sodium, about 10 mM phosphate, and about 0.6 mM sulfobetaine-16. The amount of tissue incubated in SD solution can range from about 0.1 μg to 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 2.75 mg, 5 mg, 5.25 mg, 5.5 mg, 5.75 mg, 6 mg, 6.25 mg, 6.5 mg, 6.75 mg 7 mg, 7.25 mg, 7.5 mg, 7.75 mg. 8 mg, 8.25 mg, 8.5 mg, 8.75 mg, 9 mg, 9.25 mg, 9.5 mg, 9.75 mg, 10 mg, 11 mg, 12 mg, 13 mg, 14 mg, 25 mg, 16 mg, 17 mg, 18, mg, 19, mg, 20 mg, or more. In some embodiments, the harvested tissue can be optionally initially washed prior to exposure to the solution containing SD. In some embodiments, the optional additional washes can be performed in a solution of water, phosphate buffered solutions (50 mM Na/10 mM phosphate, 1× PBS, or 100 mM Na/50 mM Phosphate), and 125 mM sulfobetaine-10. Wash times for SB-10 solution for nerve are 18 hours and 7 hours. The buffered washes can range in time from 15 minutes to 3 hours.

The tissue can be optionally washed in a saline buffer. After any optional washing, DNA can be removed by treatment with DNase 2030. DNase treatment can include incubation in a solution containing DNase. In some embodiments, the amount of DNAse can be at least 2 mL of a 75 U/mL DNAse solution. In some embodiments, the DNAse solution can contain at least 37 U

DNase/mg of tissue. In some embodiments, the solution can contain an amount of DNase that can range from about 37 U/mg of tissue to 40, 45, 50, 55 60, 65, 70, 75. 80. 85, 90, 95, or 100 U/mg of tissue. In addition to the DNAse, the DNAse solution can also contain an amount of NaCl, and an amount of MgCl₂(6 H₂O). After DNase treatment 2030, the tissue can be washed to yield the acellular tissue scaffold. The post-DNase treatment wash step can include three 1-hour washes. The wash solution can be water, PBS, or other saline buffered solution. In some embodiments, the PBS can contain 50 mM Na/10 mM phosphate, 1× PBS, or 100 mM Na/50 mM Phosphate. In some embodiments, the wash solution can contain SB-10, In other embodiments, the wash solution does not contain SB-10. In some embodiments, the amount of SB-10 can be about 125 mM. In some embodiments, the wash step can include 1 or more washes in the wash solution. The time for each wash can range from 15 minutes to 3, 7, or about 18 hours. When multiple washes are used, the solution for each wash step can be independently selected. In other words, some or all of the steps can use the same wash solution or some or all of the wash steps can use different wash solutions. Similarly, the time for each wash step can be independently selected. In other words, some or all of the washes can be performed for the same amount of time or some or all of the washes can be performed for different amount of times. The wash steps can be performed with or without agitation.

The resulting decellularized tissue (also referred to herein as an acellular tissue product or scaffold) can be immediately used (e.g. implanted) or can be lyophilized, chilled frozen, and/or otherwise stored for later processing or use. In some embodiments, the resulting decellularized tissue can undergo thermal gelation to form a hydrogel. This is discussed in greater detail elsewhere herein.

The method 2000 can produce an acellular tissue. The resulting acellular tissue can be used as a scaffold substrate for three dimensional (3D) cell culture and tissue engineering techniques. The acellular tissue can contain extracellular matrix proteins, including laminin, collagen-I, and collagen-IV. Further, the resulting acellular tissue can be delivered to a subject in need thereof to facilitate the in vivo generation and/or regeneration of cells and tissues within the subject. The resulting acellular tissue product can be formulated to be delivered to a subject in need thereof by any suitable method of deliver, including but not limited to, implantation or injection. The acellular tissue products can be enhanced by the addition of one or more other agents or compounds to the acellular tissue product, including without limitation other cells (e.g, stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. In some embodiments, the acellular tissue products can be co-administered with one or more other agents or compounds, including without limitation other cells (e.g, stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

In some embodiments, any of the acellular tissue scaffolds described herein can be administered to a subject in need thereof in the original form, as an injectable formulation, or as other derivatives of the acellular tissue. In some embodiments, the subject in need thereof has a peripheral nerve injury, spinal cord injury, spinal cord disease, amputation or otherwise suffers from degradation of a component of the spine (e.g. intervertebral disc degeneration). In other embodiments, the subject could have osteoarthritis of the hip or knee, trauma to the lungs, volumetric muscle loss, cirrhosis of the liver, or otherwise requires partial or total organ replacement.

Decellularized Tissue Hydrogels

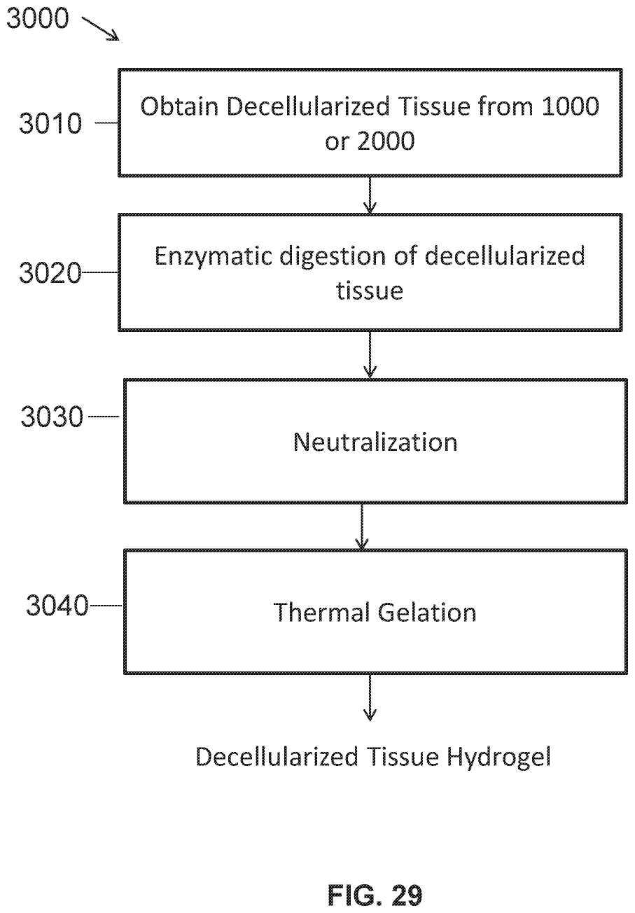

As previously discussed, the decellularized tissues produced from the apoptosis or chemical based methods described herein can undergo thermal gelation to form a decellularized tissue hydrogel. FIG. 29 shows a flow diagram outlining the general steps in a method of hydrogel formation using thermal gelation 3000. Decellularized tissue can be obtained from the chemical or apoptotic decellularization methods (1000, 2000) described herein. In some embodiments, the decellularized tissue is lyophilized. The amount of decellularized tissue can range from 0.1 milligrams to about 20 milligrams or more.

The decellularized tissue can be enzymatically digested 3020. Enzymatic digest can be completed by digesting the decellularized tissue in pepsin. In some embodiments, the digestion occurs under constant agitation. Enzymatic digestion 3020 can occur by incubating the decellularized tissue in an enzyme in solution for a period of time that can range from about 1 hour to 24, 36, 48, 64 or 72 hours or more. In some embodiments, the enzyme used in the enzymatic digestion is pepsin. The concentration of pepsin used can be about 1 to about 10 mg/mL. The enzyme can be diluted in a deionized water or hydrochloric acid solution to the about 1 to about 10 mg/mL concentration. When a hydrochloric acid solution is used, the hydrochloric acid solution can be about 0.01 M to about 0.1 M HCl. Enzymatic digestion 3020 can be performed at a temperature ranging from about 25° C. to about 37° C.

After enzymatic digestion, the digested tissue can be neutralized 3030 as needed using an appropriate solution (e.g. a sodium hydroxide solution). During or after neutralization the tissue can be further diluted using PBS to a desired tissue concentration. After neutralization or dilution the solution can be referred to as a pre-gel solution. In some embodiments, the concentration of decellularized tissue in the pre-gel solution can range from about 0.01 mg to 20 mg.

The pre-gel solution can then be used to form a hydrogel via thermal gelation 3040. Termal gelation 3040 can be performed by incubating the pre-gel solution at about 37° C. for a period of time that can range from about 15 minutes to about 60 minutes. In some embodiments the incubation period of time can be 15, 20, 25, 30, 35, 40, 45, 50, 55, or about 60 minutes. In some embodiments, the pre-gel solution can be mixed with a cargo molecule or cells that can be delivered with the decellularized hydrogel.

In some embodiments thermal gelation can occur ex vivo or in vitro. In some embodiments, it can be desirable for thermal gelation to occur ex vivo or in vitro to form a hydrogel that can be subsequently implanted into a subject. In some of these embodiments, the pre-gel solution can be put in a mold and then incubated to obtain a desired shape or size hydrogel implant. In other embodiments, it can be desirable to deliver the pre-gel solution to a subject and allow the pre-gel solution to thermally gel after delivery to the subject. In some embodiments, this can be accomplished by immediately delivering the pre-gel solution to a subject before the gelation process can take place. In other embodiments, the pre-gel solution can be kept at a temperature lower than the gelation temperature of the pre-gel solution that can delay or prevent hydrogel formation until delivery. Once delivered to the subject, a hydrogel can form inside the subject because the pre-gel solution will be exposed to a temperature of about 37° C. inside the subject.

Uses of the Decellularized Tissues and Hydrogels Thereof

The decellularized tissues (acellular tissue), pre-gel solution, or hydrogels thereof described herein can be used as a tissue scaffold and hydrogels for the repair of various soft tissue injuries. The resulting acellular tissue or hydrogel thereof can be used as a scaffold substrate for three dimensional (3D) cell culture and tissue engineering techniques. The acellular tissue, pre-gel solution, or hydrogels thereof described herein can contain extracellular matrix proteins, including laminin and collagen. Further, the resulting acellular tissue pre-gel solution, or hydrogels thereof described herein can be delivered to a subject in need thereof to facilitate the in vivo generation and/or regeneration of cells and tissues within the subject. The resulting acellular tissue product, pre-gel solution, or hydrogel thereof can be formulated to be delivered to a subject in need thereof by any suitable method of deliver, including but not limited to, implantation or injection. The acellular tissue product, pre-gel solution, or hydrogels thereof described herein can be enhanced by the addition of one or more other agents or compounds to the acellular tissue product, including without limitation other cells (e.g. stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. In some embodiments, the acellular tissue products, pre-gel solution, or hydrogels thereof described herein can be co-administered with one or more other agents or compounds, including without limitation other cells (e.g. stem cells or other progenitor cells), nucleic acids, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

In some embodiments, any of the acellular tissue scaffolds pre-gel solution, or hydrogels thereof described herein can be administered to a subject in need thereof in the original form, as an injectable formulation, or as other derivatives of the acellular tissue. In some embodiments, the subject in need thereof has a peripheral nerve injury, spinal cord injury, spinal cord disease, amputation or otherwise suffers from degradation of a component of the spine (e.g, intervertebral disc degeneration). In other embodiments, the subject could have osteoarthritis of the hip or knee, trauma to the lungs, volumetric muscle loss, cirrhosis of the liver, or otherwise requires partial or total organ replacement.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1:

Peripheral nerves were harvested from Sprague Dawley rats and decellularized according to an apoptosis-assisted protocol using 5 μM camptothecin in non-supplemented media at 37° C. for 1 day. Tissue in media without camptothecin, an apoptotic agent, was used as a control.

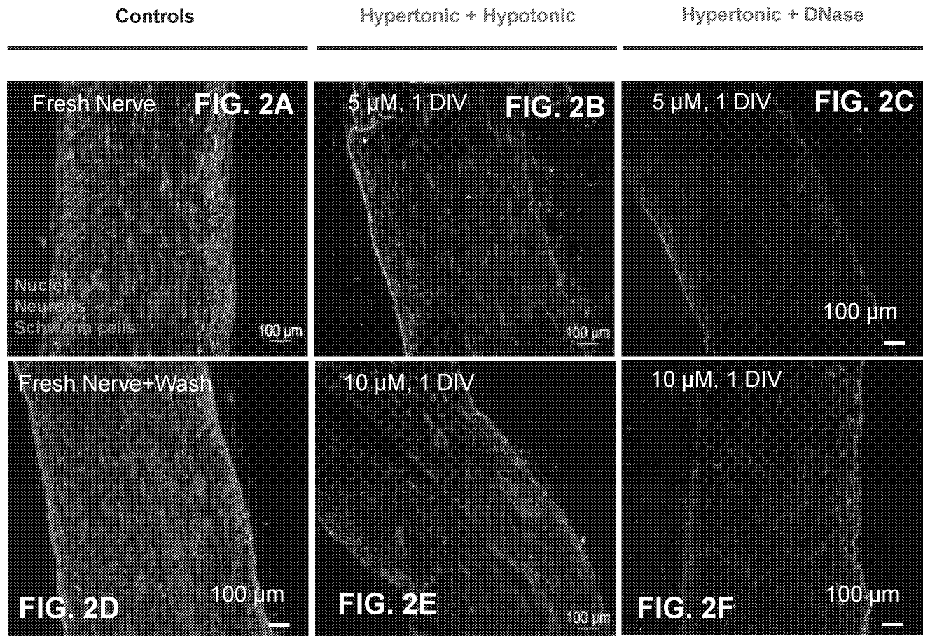

Apoptotic agent treatment was succeeded by washes in saline buffered solutions to optimize cell removal. Furthermore, the tissue was treated with 75 u/mL DNAse for 24 hours to ensure DNA removal. The degree of apoptosis, cellular removal, and matrix preservation was assessed using immunochemistry on nerve tissue sections and fluorescence imaging (FIG. 2). Antibodies used to identify apoptosis, cell removal, and tissue preservation include those against active caspase 3 (Abcam); neurofilament (RT-97, DSHB) and 5100 (Dako); and laminin (Sigma), respectively. FIG. 2A shows immunochemical staining of a fresh peripheral nerve used as a no treatment control. FIGS. 2B and 2C show nerves treated with 5 μM for 1 day, washed with a hypertonic, and either washed with a hypertonic solution or treated for 24 hours with DNAse, respectively. FIG. 2D shows staining of a fresh nerve that was subjected to the same hypertonic and hypotonic washes and represents the results of not inducing apoptosis prior to washing the tissue. FIGS. 2E and 2F show treatment with higher camptothecin (10 μM), again for 1 day, and washed similarly to tissue in FIGS. 2B and 2C. These images depict 1) apoptosis induction is necessary prior to washing to effectively remove cellular components and 2) combing apoptosis induction with washes in non-isotonic solutions achieves cellular removal of peripheral nerve tissue.

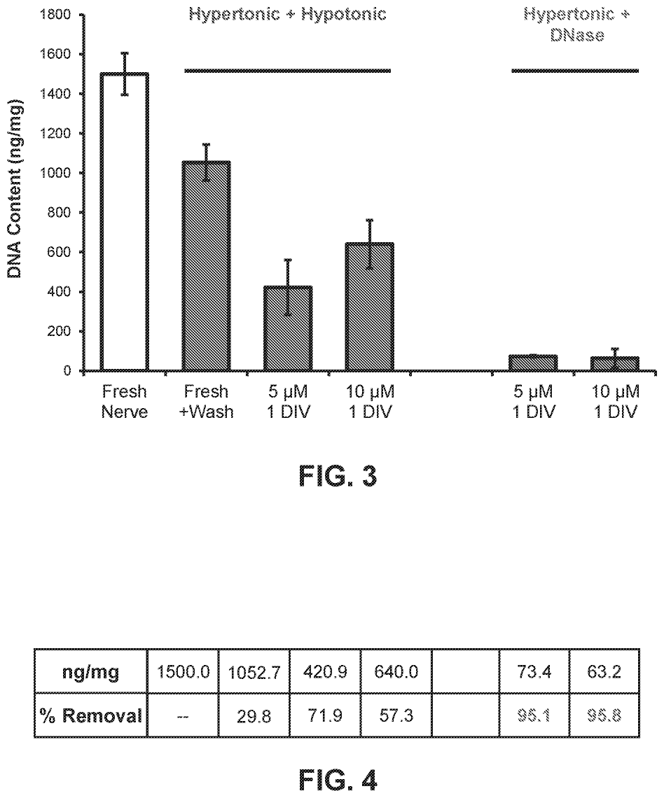

Total DNA content was quantified using a Picogreen DNA assay (Life Technologies) according to manufacturer's instructions. DNA quantification data are shown in FIGS. 3 and 4. Washing the tissue without first inducing apoptosis resulted in only a 29.8% reduction of DNA content compared to fresh nerve. Conversely, inducing apoptosis using 5 or 10 μM camptothecin yielded a 71.9 and 57.3% reduction in DNA, respectively. Replacing the hypotonic wash with DNAse treatment further reduced the DNA content, with 5 and 10 μM camptothecin treatment for 1 day resulting in a 95.1 and 95.8% reduction.

### Example 2

Peripheral nerves were harvested from Sprague Dawley rats and decellularized according to an apoptosis-assisted protocol using 10 μM camptothecin in non-supplemented media at 37° C. for 2 days. Tissue in media without camptothecin was used as a control. Apoptotic agent treatment was succeeded by washes in saline buffered solutions to optimize cell removal. The degree of apoptosis, cellular removal, and matrix preservation was assessed using immunochemistry on nerve tissue sections and fluorescence imaging. Antibodies used to identify apoptosis, cell removal, and tissue preservation include those against active caspase 3 (Abcam); neurofilament (RT-97, DSHB) and 5100 (Dako); and laminin (Sigma), respectively, Apoptosis-mediated DNA fragmentation was assessed using a terminal deoxynucleotidyl transferase dUTP nick end labeling (TUNEL) assay. Total DNA content was quantified using a Picogreen DNA assay (Life Technologies) according to manufacturer's instructions.

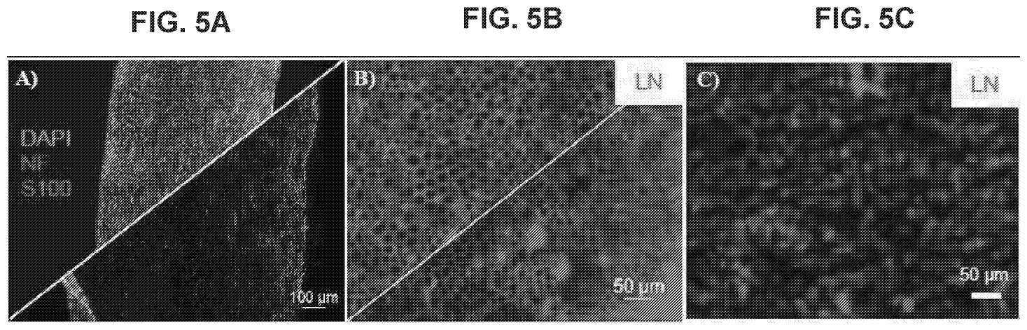

FIGS. 5A-5C demonstrate fluorescence micrographs of nerve tissue labeled for cell or basal lamina markers. For FIGS. 5A and 5B, the images are fresh nerve (top half) and apoptosis decellularized nerve (bottom half). FIG. 5A was labeled for neurons (neurofilament, green), Schwann cells (S100, red), and nuclei (DAPI, blue). FIG. 5B shows cross-sections of the basal lamina stained for laminin (red). FIG. 5C shows a representative basal lamina staining in nerve tissue decellularized according to a conventional detergent-based method. Induction of apoptosis was confirmed in treated nerve tissue by an increase in active caspase 3, an early mediator of apoptosis. Moreover, TUNEL staining revealed pervasive DNA fragmentation in treated nerves, indicating late stage apoptosis, as well. Media controls exhibited neither hallmark of apoptosis. Following induction of apoptosis, cellular proteins were easily removed using only washes in hypertonic 4× saline buffer while removal of DNA required brief washes in mildly hypotonic 0.5× saline buffer. Using this regimen, a substantial reduction in cellular and nuclear staining was achieved compared to fresh nerve. Notably, the basal lamina microstructure was nearly identical to fresh nerve and was significantly improved over alternatively processed tissue. Treatment with camptothecin was observed to be sufficient to elicit aspects of apoptosis ex vivo, including caspase 3 activation and DNA fragmentation. Moreover, induction of apoptosis was observed to enable extensive removal of cellular and nuclear components from peripheral nerve tissue using only non-isotonic buffers. Two analogs of camptothecin are considered safe by the FDA, which bolsters the clinical potential of this apoptosis-assisted decellularization method. Ultimately, the approach demonstrated here and described herein can eliminate the need for harsh lysis and chemical steps in conventional protocols and pushes decellularization technology toward achieving 3D, cell-free replicas of native tissue.

### Example 3

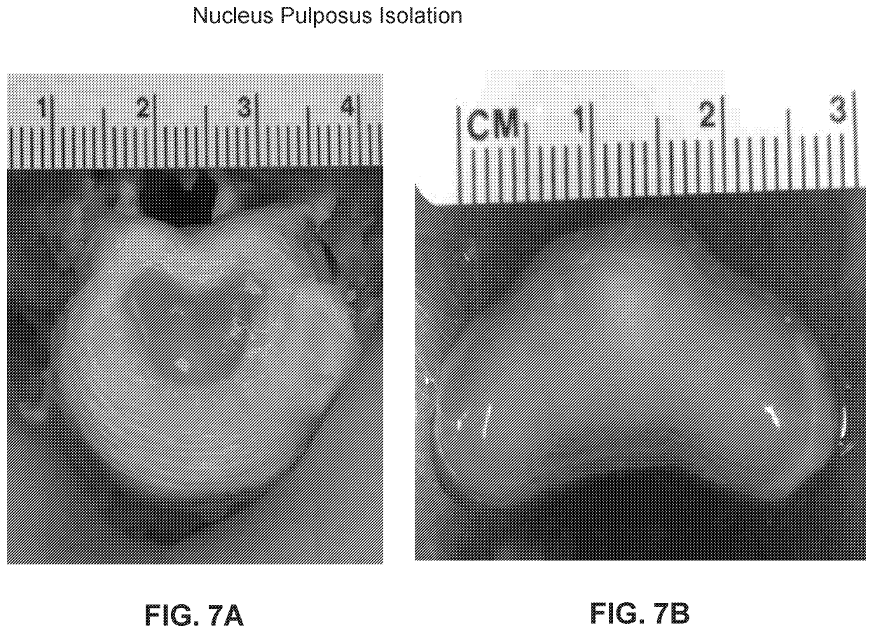

Cervical and lumbar segments were removed Yorkshire-Landrace porcine. Each motion segment was carefully dissected to extract the nucleus pulposus as shown in FIG. 7. Nucleus pulposi were decellularized according to an apoptosis-assisted protocol using 10 μm camptothecin in standard non-supplemented culture media for 24 hours. Control samples were also processed in parallel without camptothecin. After induction of apoptosis, samples were washed in a series of hypertonic and hypotonic buffers to remove apoptotic cell bodies (Table 1).

TABLE 1

| Wash Type | Method |
|---|---|
| Hypertonic | 2X PBS 30 min, 4X PBS 18 hrs, 2X PBS 30 min, 1X |
| | PBS 30 min |
| Hypotonic | 0.5X PBS 18 hrs, 1X PBS 30 min |
| Hyper-Hypo | 2X PBS 30 min, 4X PBS 18 hrs, 2X PBS 30 min, 1X |
| | PBS 30 min, 0.5X PBS 6 hr, 1X PBS 30 min |
| Hypo-Hyper | 0.5X PBS 18 hrs, 1X PBS 30 min, 2X PBS 30 min, |
| | 4X PBS 6 hrs, 2X PBS 30 min, 1X PBS 30 min |
| Hyper-DNase | 2X PBS 30 min, 4X PBS 18 hrs, 2X PBS 30 min, 1X |
| | PBS 30 min, 24 hrs DNase, 1X PBS 45 mins |
| No Wash | Fix Immediately after 24 hrs in media. |

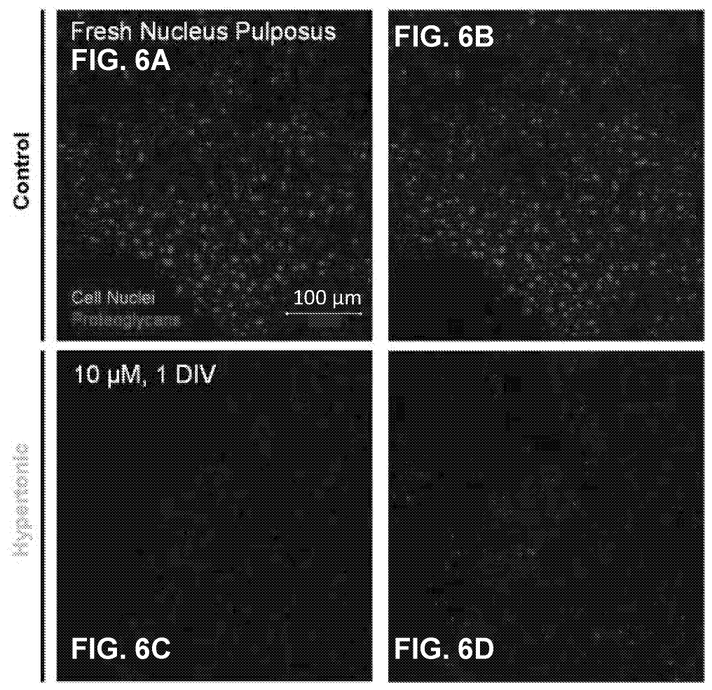

After washes, samples were processed for fixed and stained using DAPI (ThermoFisher, D1306) to detect removal of cell nuclei, and a chondroitin sulfate proteoglycan (CSPG) antibody (Sigma, C8035) to examine maintenance of key proteins. Confocal imaging was used to assess removal of cell nuclei and maintenance of CSPGs. Results demonstrated in FIGS. 6A-6D suggest the hypertonic wash and the hypertonic-hypotonic wash substantially remove cell nuclei while maintaining desired CSPGs.

### Example 4

Rat sciatic nerve was isolated from Sprague Dawley rats and decellularized using either standard detergent-based methods (those performed with Triton X-200) or apoptosis induction and tonic wash methods as detailed in Example 1 above. A rat transection model of nerve injury was performed in Lewis rats to determine efficacy of apoptosis decellularized samples compared to detergent decellularized samples, fresh harvested Lewis isograft sciatic nerves, and a sham control group. Briefly, sciatic nerve was transected and 8 mm of sciatic nerve was then removed to create a 10 mm nerve gap. After transection, one of three samples was implanted via direct suture to the nerve stumps (detergent, apoptosis, or isograft). Implants and sham controls were harvested at 4 weeks (n=3) and 8 weeks (n=6) for histological analysis. Tibialis anterior muscles were also harvested and weighed at 4 and 8 weeks to determine degree or re-innervation of a distal target, an indirect assessment of regeneration.

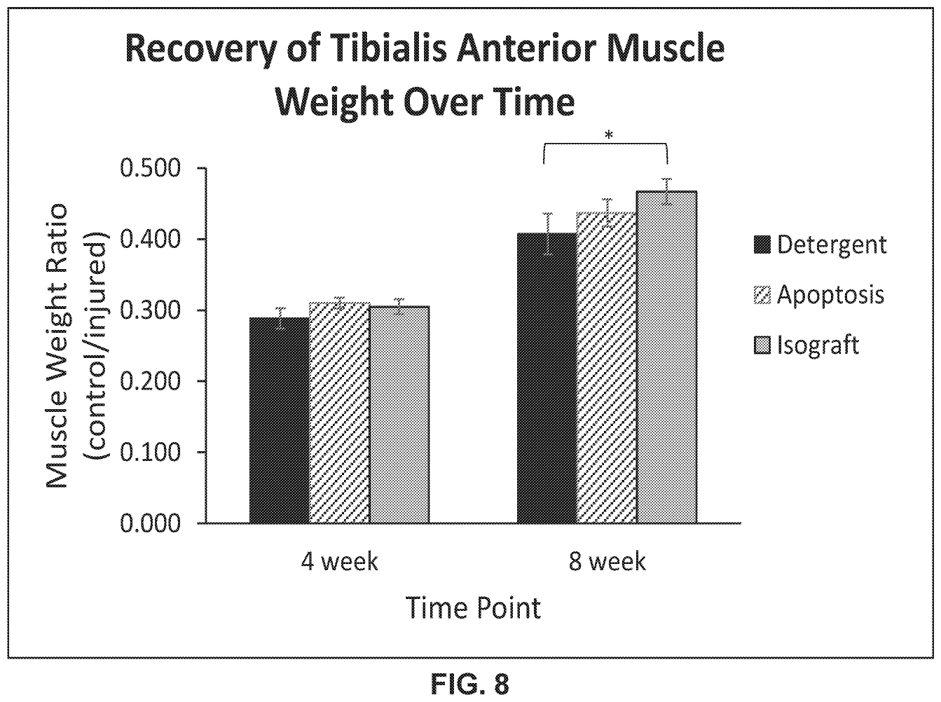

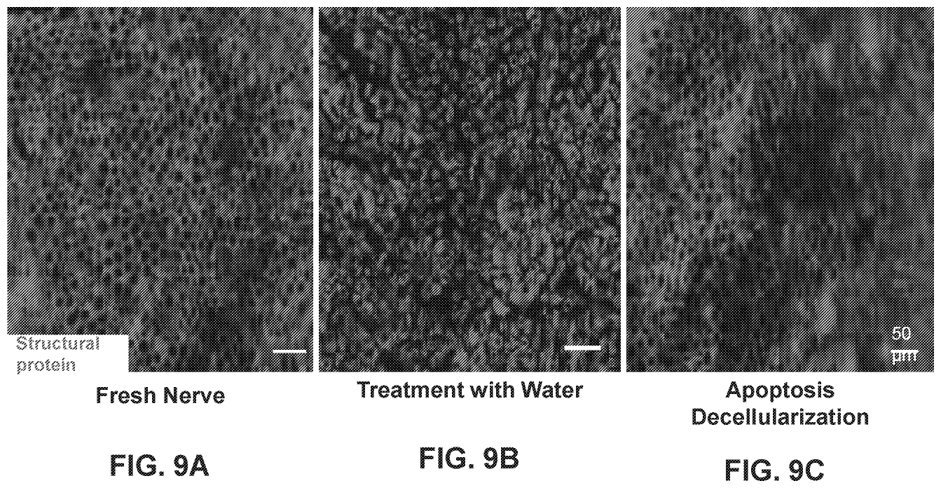

Results are shown in FIG. 8 and indicate that apoptosis decellularized samples enhance muscle recovery compared to detergent decellularized samples at both weeks 4 and 8, and are approaching levels of the isograft gold standard treatment,. Throughout the time course of the study (2, 4, 6, and 8 weeks), animals were also recorded while walking on a track to analyze alterations in gait (data not shown). Histology was used to assess the degree of regeneration between groups at both 4 weeks and 8 weeks. (data not shown).

### Example 5

Spinal cord injuries (SCIs) affect approximately 285,000 people in the U.S. alone and often result in para- or tetraplegia. Currently there is no approved treatment for SCl but numerous approaches are being researched. One of the most promising strategies is the use of decellularized biologic scaffolds. These scaffolds are highly desirable as they maintain native extracellular matrix (ECM) protein content and architecture. Scaffolds can be useful for various applications in regenerative medicine such as delivery of therapeutics and providing topographical cues. However, to prevent unwanted immune reactions, it is important to remove cellular components. SCIs commonly cause cavitation at the site of contusion, making minimally invasive injectable treatments advantageous. Cavity filling, thermally gelling scaffolds have been shown to improve regeneration and functional recovery in vivo when injected along with Schwann cells. These injectable scaffolds were developed by digesting sciatic nerves that had been decellularized via washing tissues in chemicals to lyse and remove cellular components. Although this approach facilitates the removal of cellular components, the harsh conditions cause disruption to ECM proteins (laminin and collagen) that are critical for axonal regeneration. Cell lysis results in ruptured cell membranes, which leads to the release of immunogenic intracellular elements into the extracellular environment. This undesirable aspect of current decellularization methods likely makes complete removal of cellular elements more difficult.

Surprisingly, an alternative approach to chemical detergents is induction of apoptosis. Apoptotic cells undergo organized fragmentation into small membrane bound bodies that become detached from the ECM. This method does not require the use of chemical detergents and allows for shorter wash times, increasing preservation of ECM. Disclosed herein is the development of an injectable, thermally gelling scaffold derived from rat sciatic nerve decellularized via apoptosis rather than harsher previously employed methods. As demonstrated elsewhere herein decellularization via apoptosis can adequately remove cellular content while preserving crucial ECM proteins. By tuning digestion conditions such as temperature, tissue concentration, digest duration and acid type, thermal gelation decellularized nerves have been successful. These thermally gelled these scaffolds can be used to establish localized and controlled delivery of therapeutics to stimulate axonal outgrowth and functional recovery in a rat SCI model, and ultimately in human patients.

This Example can demonstrate injectable tissue hydrogels formed from thermally gelled decellularized tissue that has been made using an apoptotic processing methodology described herein. The thermally gelled tissue hydrogel scaffolds can be injectable. The injectable tissue hydrogels described herein can be seeded with cells, such as Schwan cells, or other cells or co injected with cells, such as Schwan cells. Typical tissue scaffold preparation involves washing tissues (such as nerves) in detergents, releasing intracellular components throughout the matrix. Although this approach facilitates the removal of cellular components, the harsh conditions cause undesired disruption to ECM proteins (collagen and laminin) which are critical for the regenerative environment.

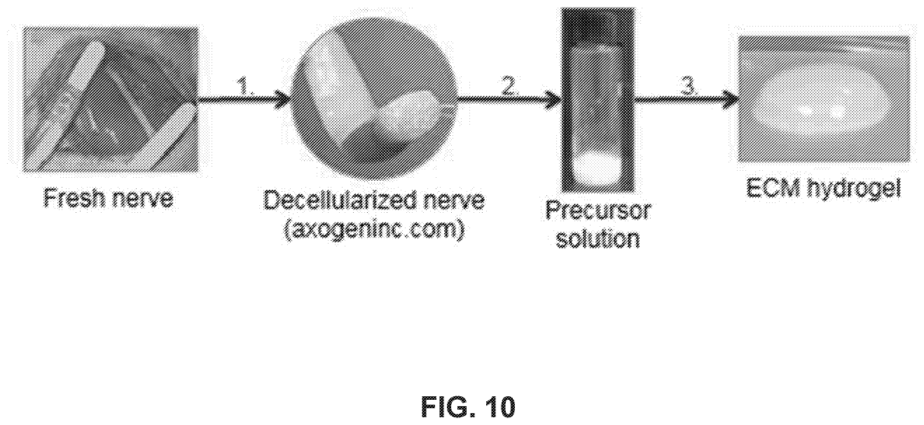

The general process for preparing a tissue hydrogel is shown in FIG. 10, which shows a flow diagram demonstrating the general steps in forming a decellularized tissue hydrogel using apoptotic or chemical (e.g. SD based) decellularization followed by enzymatic digestion and thermal gelation. After the fresh tissue is harvested, the tissue can be decellularized using chemical (e.g. SD-based) and apoptotic decellularization process (1.) to obtain a decellularized tissue. Enzymatic and further processing (2) can be performed to yield a solution that can undergo thermal gelation (3.) to form a tissue hydrogel (ECM hydrogel).

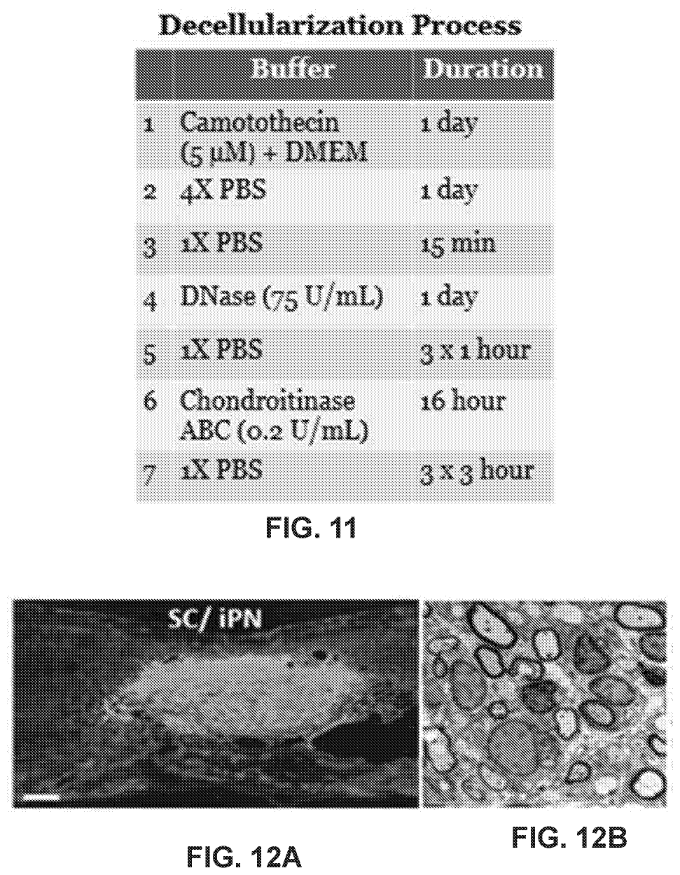

Briefly, the injectable tissue hydrogels of this Example were generated by digesting rat sciatic nerves that had been decellularized by inducing cell necrosis (or apoptosis). Apoptosis can cause cells to form small apoptotic bodies containing cellular components, which can be more easily removed from the matrix. This approach has the potential to enhance matrix preservation and bioactivity, thus having a major impact on tissue engineering. In this Example, rat sciatic nerves were first harvested and decellularized by inducing apoptosis (described elsewhere herein and e,g. FIG. 11). Washing in camptothecin can induce apoptosis. Hypertonic washing (e,g. 4× PBS) can aid in cell removal. DNase treatment can degrade residual DNA content. Treatment with Chondroitinase ABC can be used to remove inhibitory proteoglycans. After the decellularization process, the resulting decellularized tissue can undergo enzymatic processing and further processing to balance pH where necessary. In some cases the decellularized tissue can be lyophilized prior to enzymatic digestion. In this particular example, pepsin was used to digest tissue at an acidic pH. After about 64 of enzymatic digestion, the solution can be neutralized and isotonically balanced. The solution was further diluted with PBS. In some embodiments, the solution was diluted to 1/9 th of its volume in 10× PBS. After this, thermal gelation was performed to cross link various remaining components of the ECM. Thermal gelation was performed by incubating the resulting solution from previous steps at 37° C. for about 30 minutes.

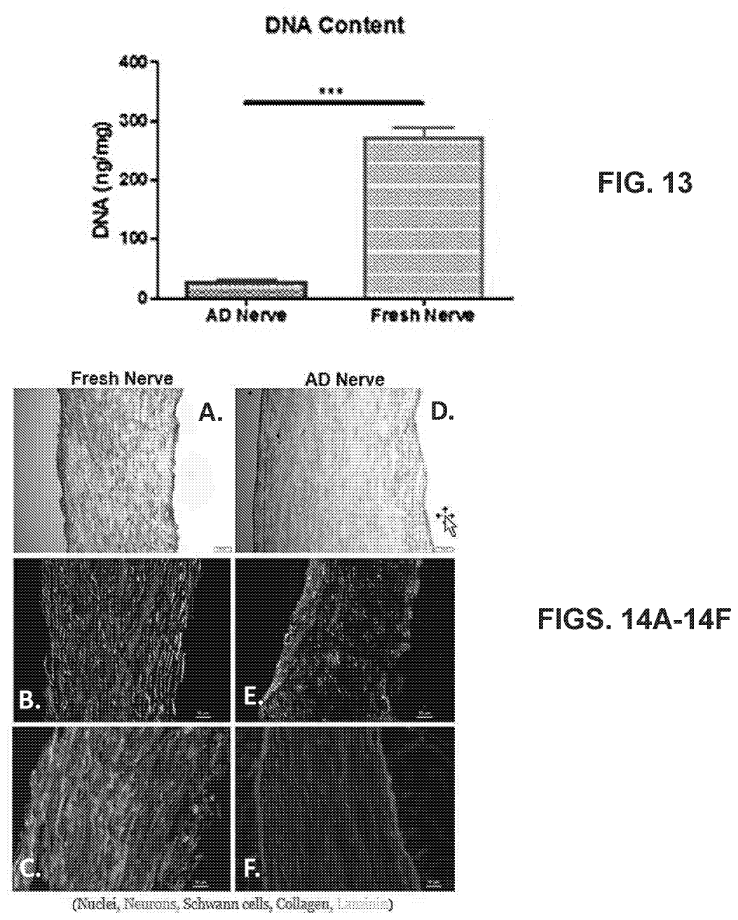

Preliminary studies using a TUNEL assay showed enhanced apoptosis in samples treated with camptothecin. DNA removal was quantified using a QuantiFluor dsDNA kit, which demonstrated removal of greater than about 90% DNA without the use of harsh chemicals. See e.g. FIG. 13. Immunohistochemistry analysis suggests removal of cellular components (S-100 and DAPI) while preserving ECM proteins collagen and laminin (FIGS. 14A-14F),The tissue hydrogels (also referred to in this context as injectable peripheral nerve) were prepared from rat sciatic nerve and injected along with Schwan cells into the spinal cord. The results are shown in FIGS. 12A-12B.

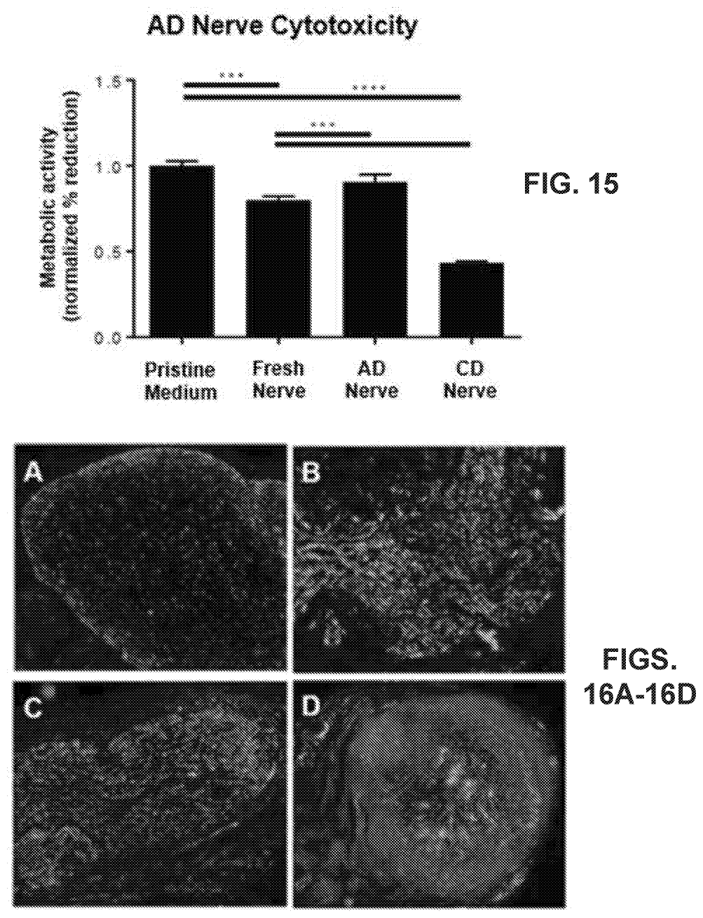

FIG. 15 shows a graph that can demonstrate cytotoxicity of Schwann cells cultured in media eluted from apoptosis decellularized (AD) nerve tissue, chemically decellularized (CD) nerve tissue, fresh nerve tissue, and fresh media. AlamarBlue reduction suggested that only AD nerve and fresh media groups did not alter metabolic activity of the cultured Schwann cells.

FIGS. 16A-16D show fluorescent microscopy images that can demonstrate subcutaneous implantation of decellularized nerves into rats allowed for analysis of an immune reaction.

Macrophage (green) and nuclear (blue) staining can demonstrate that macrophage infiltration was lowest in AD nerves (FIG. 16A) as compared to CD nerves (FIG. 16B), isografts (FIG. 16C), and allografts (FIG. 16D).

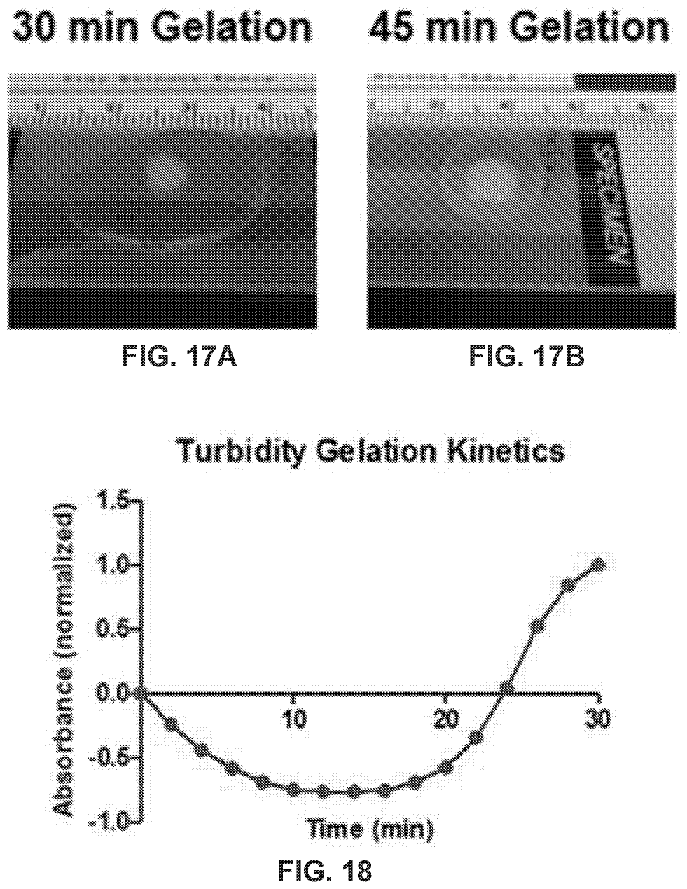

FIGS. 17A-17B show the effect of different thermal gelation conditions. Thermal gelation was performed via enzymatic digestion of AD nerves followed by incubation of a precursor solution at about 37 for up to about 60 minutes (data shown here is at 30 minute incubation and 45 minute incubation. Gelation was confirmed qualitatively by placing gels in 1× PBS. Precursor solutions containing AD nerve tissue incubated for 30, 45 or 60 minutes produced gels that maintained their shape indicating thermal gelation in these cases was achieved.

FIG. 18 shows a graph that can demonstrate the turbidity gelation kinetics of the tissue hydrogels formed by measuring the absorbance (405 nm) of one sample over time while incubating at about 37° over time as gelation occurs, The graph in FIG. 18 can suggest that full gelation can occur shortly after about 30 minute incubation.

### Example 6

In addition to serving as a scaffold, the thermally gelled tissue hydrogels can be used as a delivery scaffold for therapeutics and other compounds to a specific site within a subject. One of the main physical and molecular barriers to regenerating axons after spinal cord injury (SCI) is the formation of a glial scar. Chondroitin sulfate proteoglycans (CSPGs) are among the most abundant proteoglycans and contribute to the glial scar formation. Several, in vivo and in vitro studies have provided evidence that the use of chondroitinase ABC (ChABC) enzyme can help promote axonal regeneration and functional recovery by enzymatic breakdown of scar components. However, ChABC delivery is severely limited due to its unstable nature which demands repeated intrathecal injections causing discomfort and posing risk for recurrent infections. These drawbacks can be overcome by using drug delivery systems which offer controlled release at the site of injury. The iPN hydrogels described herein can be used as a drug delivery system for therapeutics, such as chABC, as they are biocompatible, and mimic the mechanical properties and ECM protein composition of native neural tissue. It will be appreciated that ChABC here is demonstrative of delivery of a cargo molecule or other cargo molecules that can bind to or otherwise interact with an ECM or other component of the thermally gelled tissue by the thermally gelled tissue hydrogels described herein.

Briefly, a ChABC fusion protein with Galectin 3 (ChABC-Gal_3) was produced in E. coli and purified using immobilized metal affinity chromatography. The purity of the protein was confirmed by gel electrophoresis. Tests were performed to confirm the bioactivity of ChABC-Gal_3 unit by allowing ChABC fusion protein to breakdown chondroitin sulfate chains and proteoglycan decorin. Galectin proteins can bind non-covalently to ECM proteins; collagen and laminin present in the hydrogel matrix and to the CSPGs present at the SCI site. Therefore, release of ChABC from iPN hydrogels can be controlled based on the binding affinity between Gal_3 and ECM proteins in the hydrogel matrix and the released protein can be localized based on Gal_3 interaction with CSPGs. Preliminary experiments using model fluorescent compounds and hydrogel were successful in obtaining sustained release of GFP-Gal_3 fusion protein over 7 days compared to GFP control. Currently, experiments are in progress to obtain longer release profiles for up to 4 weeks by preparing trimer fusion protein (GFP-3Gal_3). ChABC-Gal_3 encapsulated iPN hydrogels can be delivered to in an in vivo environment using a rat SCI model.

### Example 7

This Example describes, inter alia, a chemical decellularization protocol developed for tissues, as demonstrated by performance on peripheral nerve tissue. The protocol described in this Example differs from the previously established peripheral nerve decellularization protocol (developed by Hudson et al 2004¹ and utilized by Cornelison et al 2018² and Cerqueira et al.³) in that a 24 hour Triton X-200 wash is replaced by a 2 hour, 3% sodium deoxycholate (SD) wash and inclusion of an additional 3 hour deoxyribonuclease (DNAse) incubation and three 1-hour buffer washes after treatment with DNase, The chemical decellularization protocol described in this Example can effectively remove cellular components while retaining the extracellular matrix (ECM) proteins (e.g. laminin and collagens (e.g. at least collagen- I, collagen- and IV, and laminin. This produces an acellular peripheral nerve scaffold that can be used as a peripheral nerve graft or could be solubilized to form an injectable hydrogel for spinal cord injury or other injuries depending on starting tissue type.

A previous protocol for the creation of thermal gelation hydrogels from the Triton X-200 decellularized peripheral nerve developed was used as a starting point to optimize the solubilization and hydrogel formation after the new SD decellularization process. According to Cornelison's protocol, 10 mg of porcine pepsin was dissolved in 1 mL of 0.1 M hydrochloric acid (HCl) for a final concentration of about 10 mg/mL. The Triton X-200 decellularized nerves were weighed and cut into 1-2 mm pieces via microscissor into a small vial with a magnetic stir bar.

The pepsin solution was diluted in deionized water to a concentration of 1 mg/mL and enough of the resulting solution was added to the tissue such that the total tissue concentration was 20 mg/m, The tissue was then incubated in the pepsin solution under constant agitation on a stir plate for about 64 hours. After this solubilization, the solution was neutralized with 1 M sodium hydroxide (NaOH) and 10× phosphate buffered solution (PBS) until the pH of the pre-gel solution was approximately 7.4.

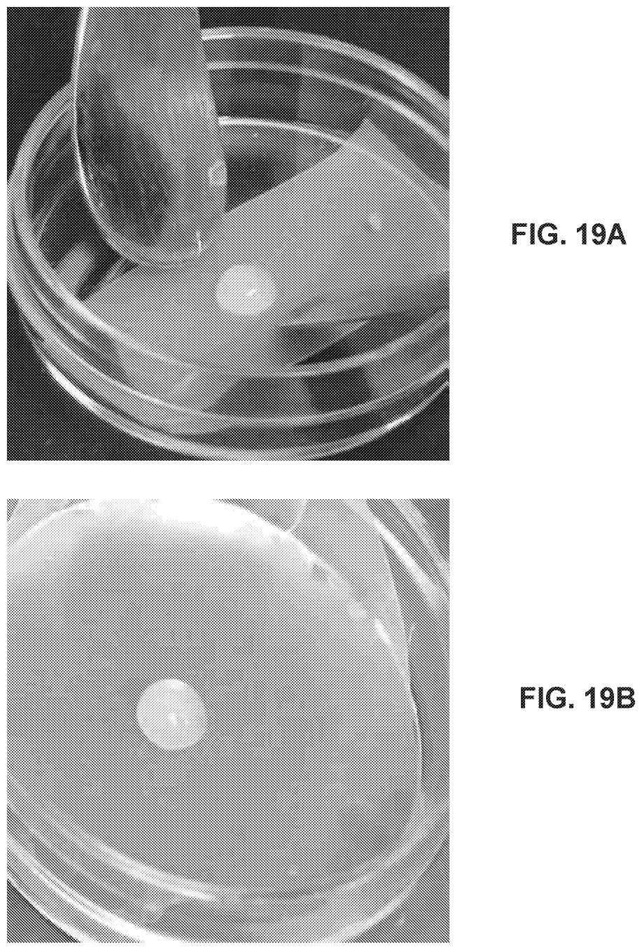

The original protocol was followed using SD (instead of Triton X-200) decellularized nerves with a final tissue concentration of 20 mg/mL. However after the 64 hours, the tissue was not entirely solubilized and the pre-gel solution was too viscous to be used in an injectable form. To account for this, we then tested a lower concentration of tissue and incubated for a longer time period for facilitate further solubilization. The tissue concentration was decreased to 15 mg/mL tissue, and the incubation time in the pepsin solution was extended to about 72 hours. The pre-gel solution was neutralized as outlined in Cornelison's original protocol and after incubating at 37° C. for 15 minutes, a robust tissue hydrogel was formed (FIGS. 19A-19B). Initial neutralization was carried out at room temperature and caused some apparent gelation, causing inhomogeneity in the pre-gel solution. The gelation of the solubilized tissue at room temperature was overcome by keeping the pre-gel solution on ice or at 4° C. during neutralization. Further, the formation of thermal hydrogels from the decellularized peripheral nerves, 5 mg/mL, 7.5 mg/mL, and 10 mg/mL tissue concentrations were evaluated (summary in Table 1).

TABLE 1

| Tissue | | | |
|---|---|---|---|
| Concentration | | Dissolution | Injectable (Standard |
| (mg/mL) | Gelation? | in PBS? | Micropipette Tip) |
| 5 | N | N | N |
| 7.5 | Y | Y | Y |
| 10 | Y | Y | Y |
| 15 | Y | Y | N |
| 20 | N | N | N |

Only 7.5 mg/mL, and 10 mg/mL tissue concentrations were able to form gels and maintain the structure after a buffer solution was added (FIG. 19A and 19B, respectively) and the viscosities for 7.5 mg/mL and 10 mg/mL tissue concentrations were low such that it could be possible to use them in an injectable form. FIGS. 19A-19B show tissue hydrogels formed from initial tissue concentrations (FIG. 19A) 7.5 mg/mL, (FIG. 10B) 10 mg/mL, and following 15 minutes in 37° C.

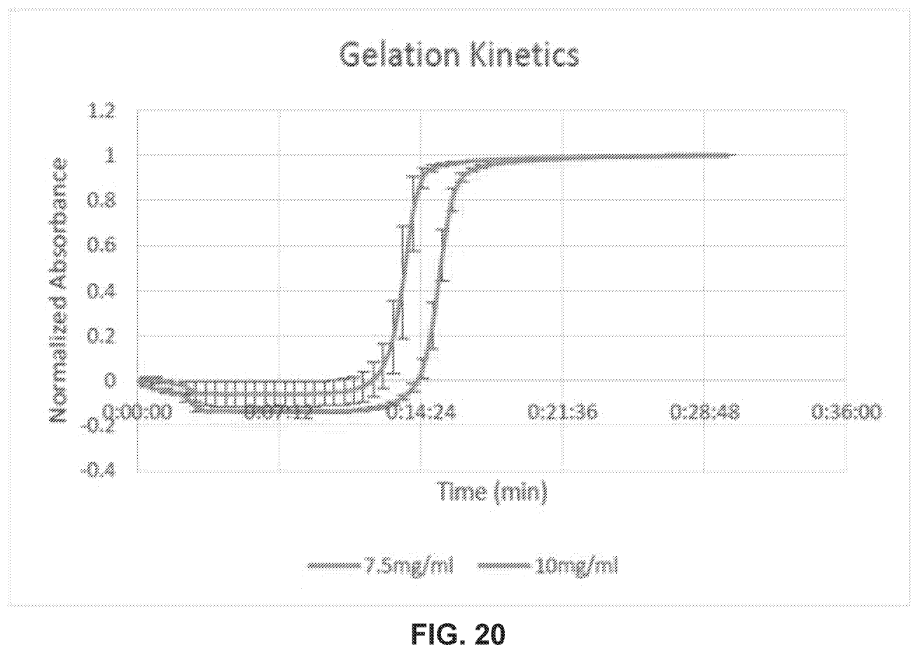

Further characterization of the hydrogels formed have been conducted including, These include gelation kinetics and gelation kinetics for both concentrations. Results are summarized in FIG. 20.

TABLE 2

| | Time to 50% | Time to 95% | Slope of | |
|---|---|---|---|---|
| Concen- | gelation | gelation | the linear | Lag time |
| tration | (t½) | (t95) | region(s) | (tlag) |
| 7.5 mg/mL | 15 min 30 sec | 18 min | 738.53 | 14 min 30 sec |
| 10 mg/mL | 13 min 45 sec | 15 min 30 | 639.25 | 12 min 30 sec |
| | | sec | | |

FIG. 20 shows a graph that can demonstrate the normalized absorbance of the sample which depicts the formation of the hydrogel when incubated at 37° C. Hydrogels with a concentration of 10 mg/mL consistently exhibited faster gelation rates (t1/2 about 13 minutes 45 seconds) than those with tissue concentrations of 7.5 mg/mL (t1/2 about 15 minutes 30 seconds). A set of gelation parameters has been developed (Table 1) including the time to 50% and 95% gelation, slope of the linear region (s), and lag time until gelation (flag).

FIG. 29 shows a flow diagram demonstrating general steps to form a decellularized tissue hydrogel from the decellularized tissues made from the apoptosis or chemical decellularization methods described herein.

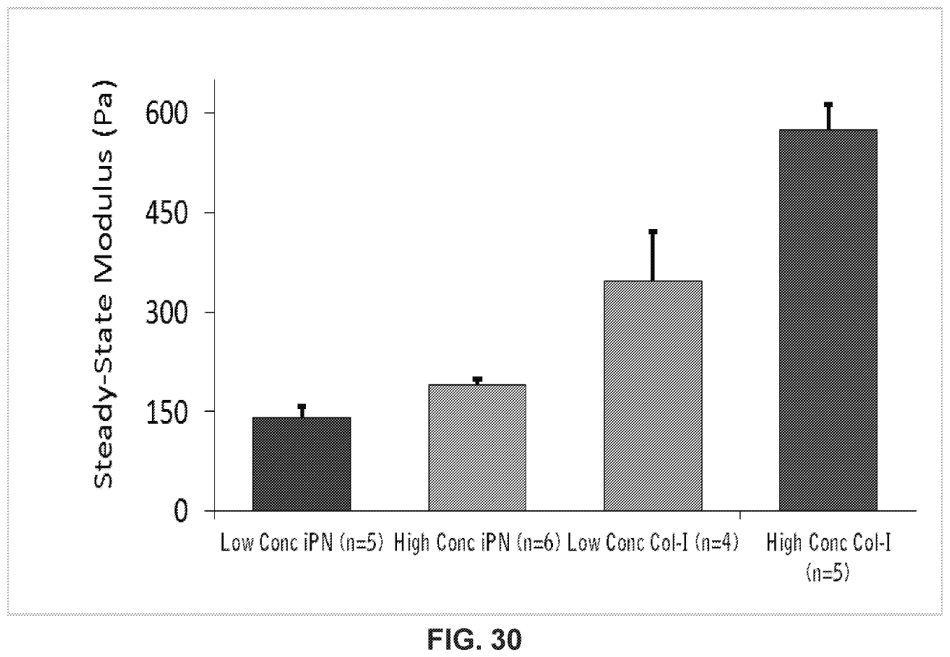

FIG. 30 shows a graph that can demonstrate rheological properties of an iPN. This graph can demonstrate the shear modulus for low and high concentration iPN (7.5 and 10 mg/ml). These are shown against pure collagen-I hydrogels at similar concentrations for reference. iPN hydrogels derived using the sodium deoxycholate decellularization method.

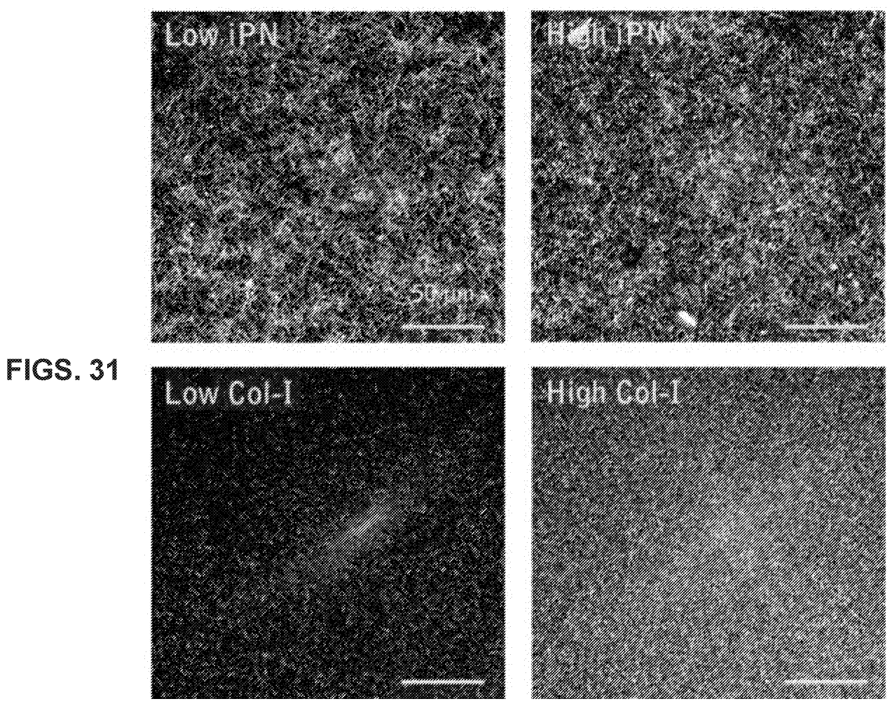

FIG. 31 shows a panel of confocal reflectance images of low and high iPN concentration hydrogels (7.5 and 10 mg/ml) and these are shown against pure collagen-I hydrogels at similar concentrations for reference. iPN hydrogels derived were from the new sodium deoxycholate decellularization method.

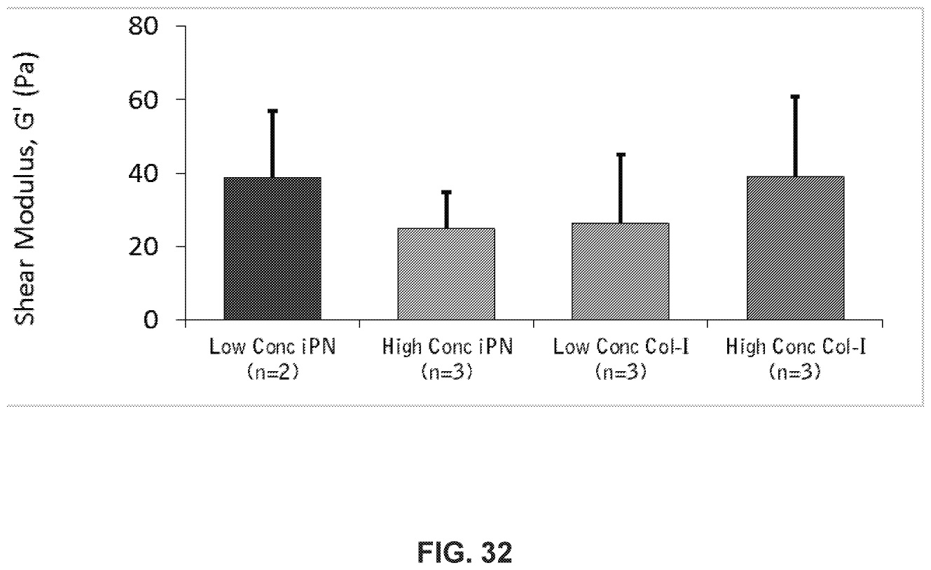

FIG. 32 shows a graph that can demonstrate the shear modulus for low and high concentration iPN (7.5 and 10 mg/ml), These are shown against pure collagen-I hydrogels at similar concentrations for reference. iFN hydrogels derived using the sodium deoxycholate decellularization method.

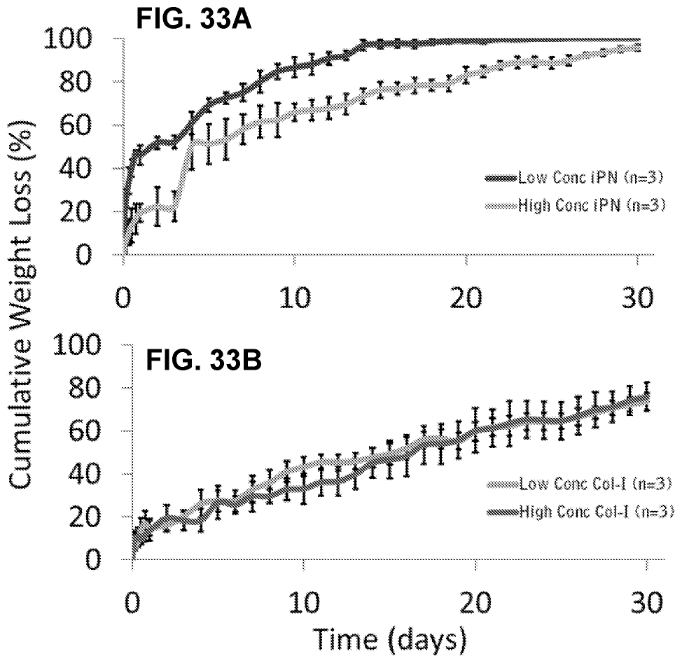

FIGS. 33A and 33B show graphs that can demonstrate =in vitro degradation rates shown as cumulative weight loss (%) over the course of 30 days for low (7.5 mg/ml) and high (10mg/ml) iPN hydrogels derived from the sodium deoxycholate decellularization method. These are shown against pure collagen-I hydrogels at similar concentrations for reference.

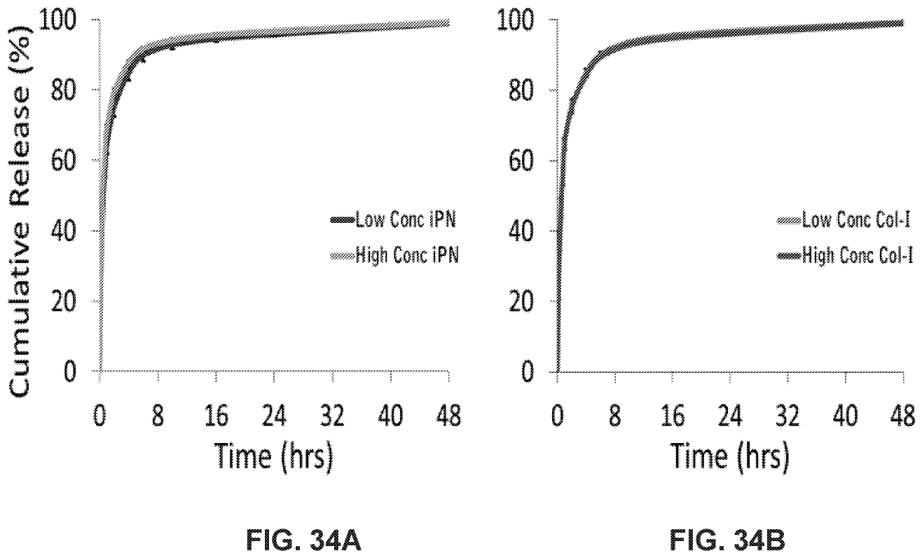

As noted elsewhere herein, the decellularized tissue hydrogels can be used to deliver a payload, such as a therapeutic or other cargo molecule. FIGS. 34A-34B show graphs that can demonstrate Pseudo-drug (FITC-DEXTRAN) release profiles for low and high concentration iPN (7.5 and 10 mg/ml) generated from the sodium deoxycholate decellularization method. These are shown against pure collagen-I hydrogels at similar concentrations for reference.

### REFERENCES FOR EXAMPLE 7

1. Hudson T W, Liu S Y, and Schmidt C E. Engineering an improved acellular nerve graft via optimized chemical processing. Tissue Engineering. 2004. 10(9-10): 1346-1358.

2. Cornelison R C, Gonzalez-Rothi E J, Porvasnik S L, Wellman S M, Park J H, Fuller D D, and Schmidt C E. Injectable hydrogels of optimized acellular nerve for injection in the injured spinal cord. Biomedical Materials. 2018. 13(3): 034110.

3. Cerqueira S R, Lee Y S, Cornelison R C, Mertz M W, Wachs R A, Schmidt C E, and Bunge M B. decellularized nerve matrix supports Schwann cell transplants and axon growth following spinal cord injury. Submitted. 2018.

4. Medberry C J, Crapo P M, Siu B F, Carruthers C A, Wolf M T, Nagarkar S P, Agrawal V, Jones K E, Kelly J, Johnson S A, Velankar S S, Watkins S C, Modo M, and Badylak S F. Hydrogels derived from central nervous system extracellular matrix. Biomaterials. 2013. 34(4): 1033-1040

### Example 8

As described in Example 7, a chemical decellularized process can be used to prepare decellularized tissue products and hydrogels thereof. This Example demonstrates chemically decellularized lung tissue based upon the same protocol described in Example 7.

Briefly, rat pup lungs were chemically decellularized with an SD-based protocol (see e.g. Example 8) and lyophilized. After lyophilization, the lungs were digested for at least 72 hours under constant agitation in about 1 mg/mL pepsin in 0.01 M HCl. The digest solution was then neutralized to a pH of about 7.4 using 1 N NaOH and 10X PBS to form a pre-gel solution. The resulting pre-gel solutions were then incubated at about 37° C. for at least 15 minutes to induce thermal gelation and hydrogel formation. The extent of hydrogel formation was then assessed. Gelation, integrity, and injectability results are summarized in Table 3.

TABLE 3

| Tissue | | Integrity | Injectable? |
|---|---|---|---|
| Concentration | | maintained | (standard Micro- |
| (mg/mL) | Gelation? | in PBS? | pipette Tip) |
| 7.5 | Yes | Yes | Yes |
| 10 | Yes | Yes | Yes |
| 15 | No | No | No |

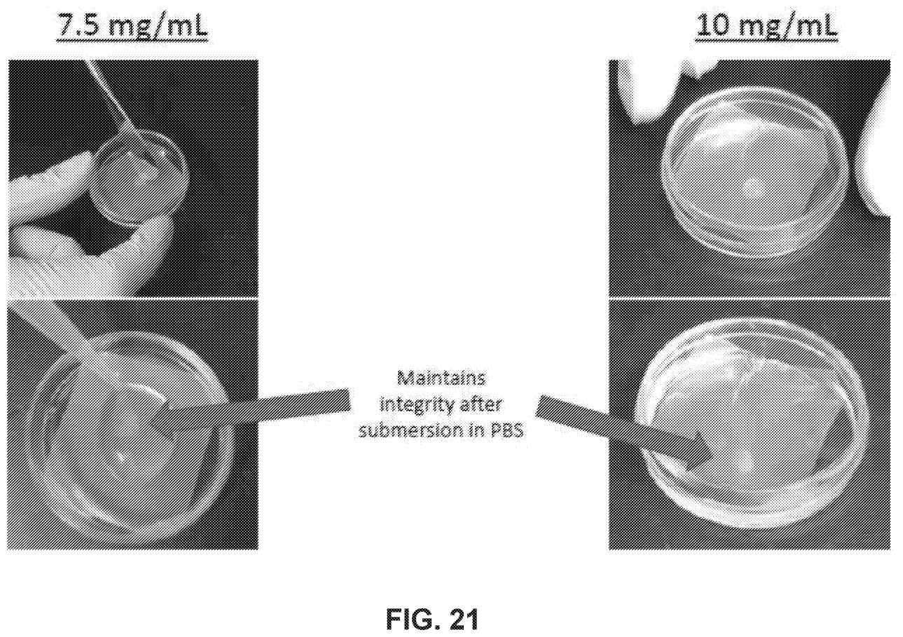

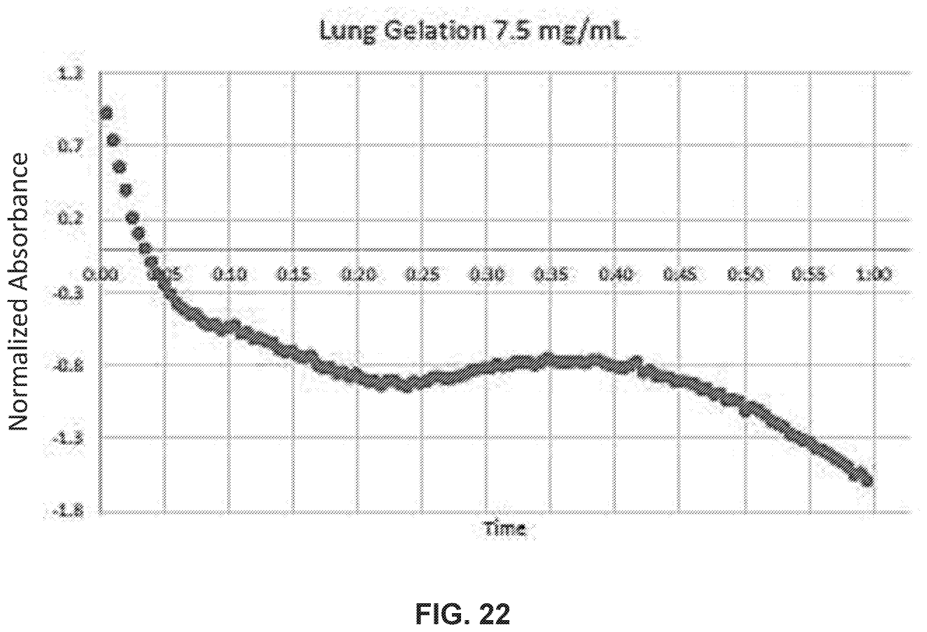

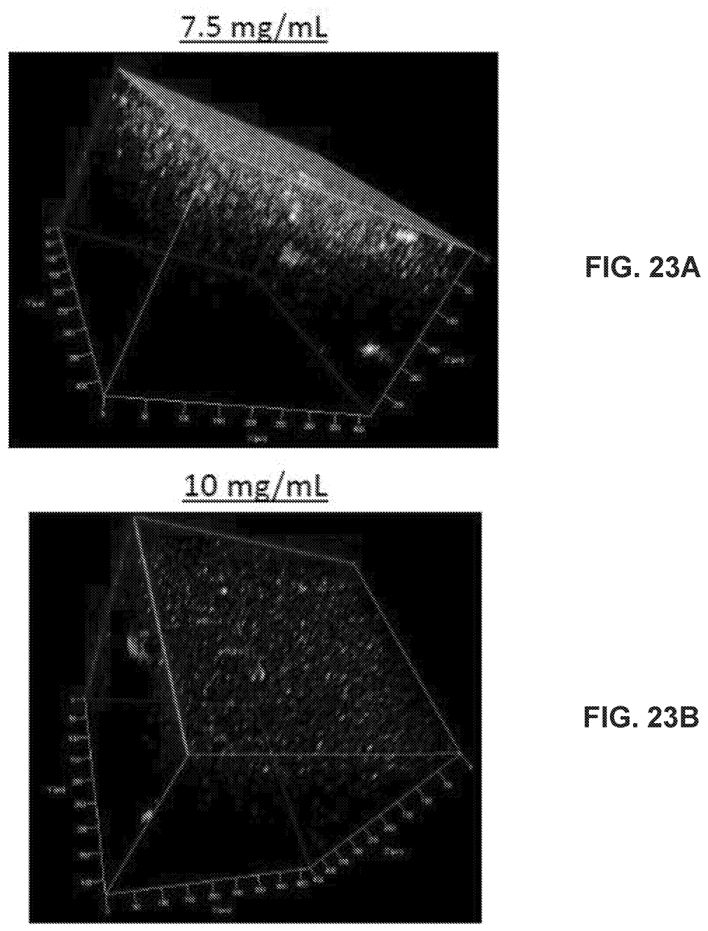

Images of the hydrogels formed from chemically decellularized lung tissue and results from integrity tests (submersion in PBS) are shown in FIG. 21. FIG. 22 shows a graph that can demonstrate the gelation kinetics from chemically decellularized lung tissue. The absorbance at 405 nm was measured over time during gelation incubation for 60 minutes. Based on this kinetic profile, gelation appears to be via a non-collagen gelling mechanism. FIGS. 23A-23B shows results from confocal collagen reflectance from chemically decellularized lung tissue. These images can demonstrate that despite the kinetic profile indicating a non-collagen-based gelling mechanism, collagen is still homogenously distributed throughout the hydrogels.

### Example 9

Example 8 described decellularized lung tissues and hydrogels thereof prepared using a chemical decellularization methods. Described elsewhere herein are apoptotic based decellularization methods. This Example describes decellularization of lung tissue using an apoptotic based method, such as those described herein, and hydrogels thereof.

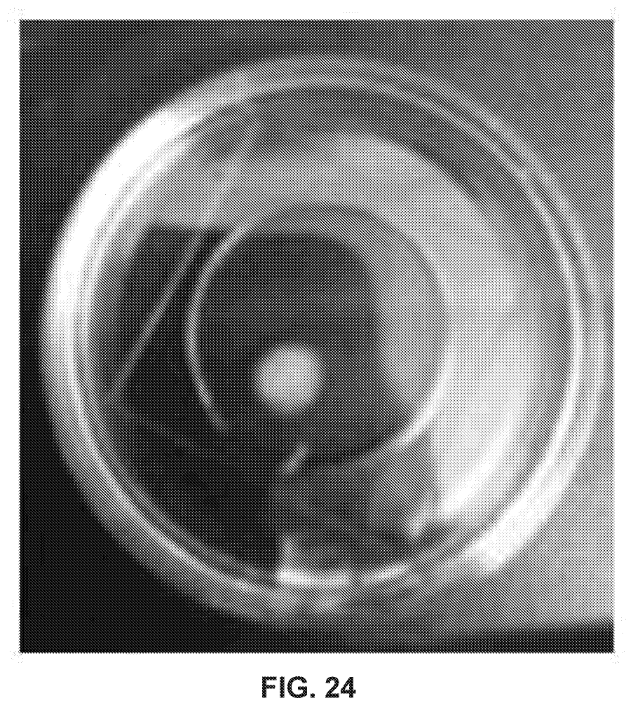

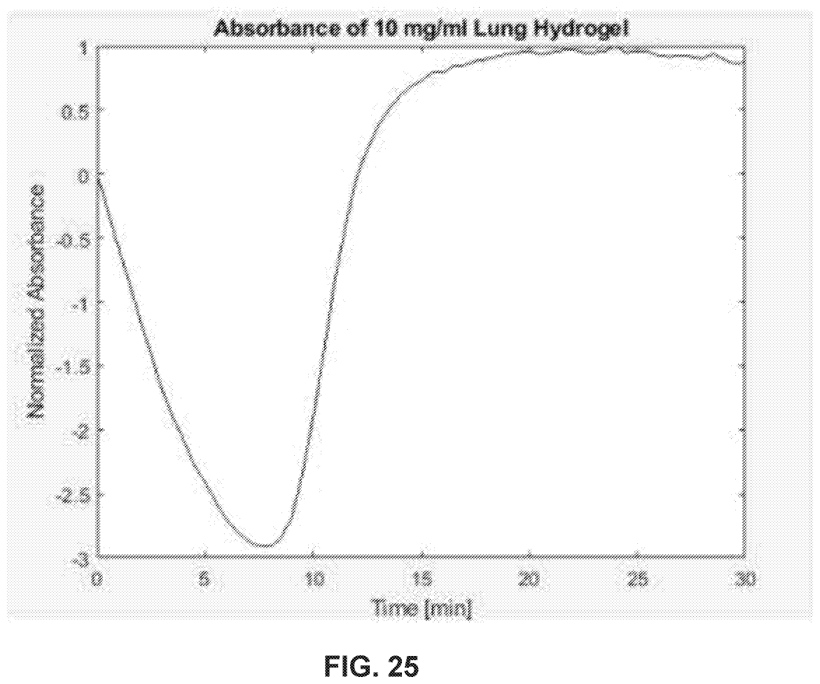

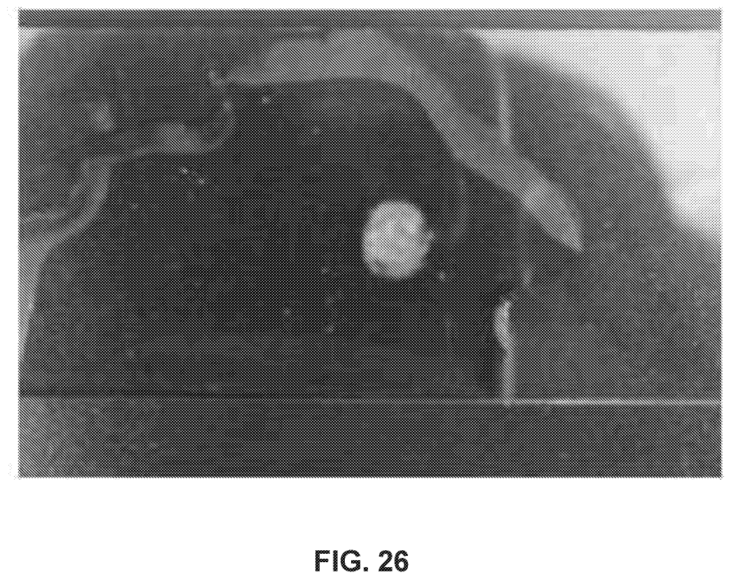

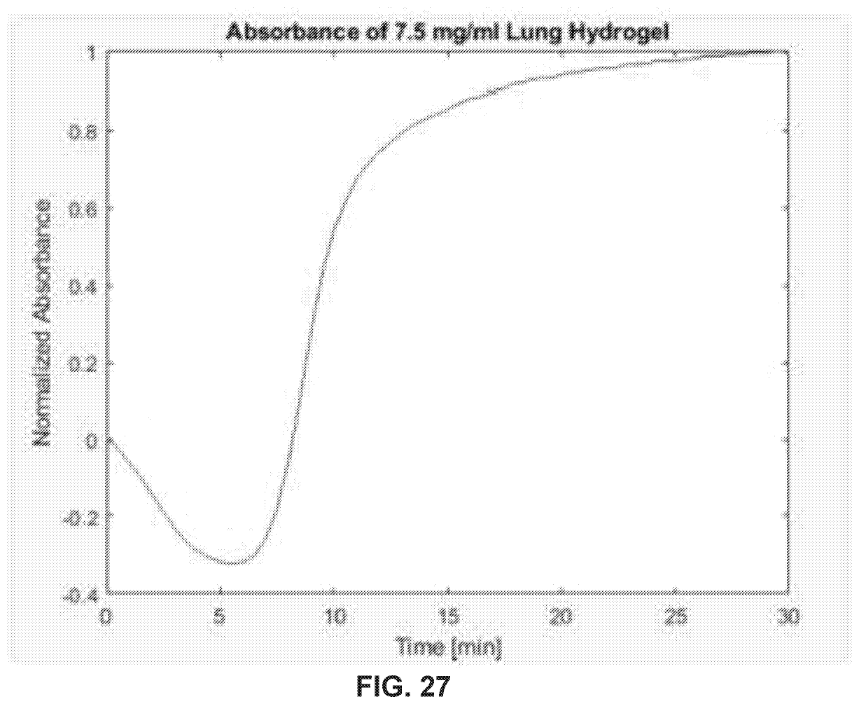

Briefly, adult rat lung tissue was harvested after blanching with PBS perfusion via the right ventricle and several lung tissue pieces were obtained using a 4 mm biopsy punch. The lung tissue pieces were subjected to a 20 μM camptothecin in basal media for about 48 hours at about 37° C. under agitation. The tissues were rinsed in 4× PBS at room temperature under agitation (12 hours×4). The tissues were then soaked in 125 mM SB-10 at RT under agitation for about 7 hours. The tissues were then rinsed again in 1× PBS at room temperature under agitation (1 hour× 3. The tissue was then incubated in DNase solution for 24 hours without agitation at room temperature. After DNase treatment, the tissue was rinsed in 1× PBS at room temperature under agitation (1 hour×3). The resulting decellularized product was lyophilized, cut into 1-2 mm pieces and digested in 1 mg/mL pepsin in 0.01 M HCl at a concentration of 10 or 7.5 mg of tissue per mL pepsin-HCl solution at room temperature for about 72 hours. After enzymatic digestion, the resulting solution was neutralized using 1 M NaOH, followed by the addition of 1/10 total volume of 10× PBS. After neutralization, hydrogels were formed by thermally gelling the pre-gel solution formed after neutralization. Thermal gelation was completed by incubating the pre-gel solution at about 37° C., for about 20-40 minutes. FIG. 24 shows an image of a lung tissue hydrogel formed using 10 mg/mL of tissue for gelation as described above. FIG. 25 shows a graph the gelation kinetics of the 10 mg/mL tissue hydrogel shown in FIG. 24. FIG. 26 shows an image of a lung tissue hydrogel formed using 7.5 mg/mL of tissue for gelation as described above. FIG. 27 shows a graph of the gelation kinetics of the 7.5 mg/mL tissue hydrogel. In both cases, the gelation kinetics were obtained by measuring absorbance at 405 nm of the tissue during gelation at 37° C. for about 30 minutes.

1. A decellularized tissue hydrogel comprising:
decellularized tissue, wherein the decellularized tissue contains native extracellular matrix proteins,
wherein the decellularized tissue is cross-linked to form the hydrogel.

2. The decellularized tissue hydrogel of claim 1, further comprising a therapeutic agent.

3. The decellularized tissue hydrogel of claim 2, wherein the therapeutic agent is a population of cells, a polynucleotide, an amino acid, a peptide, a polypeptide, an antibody, an aptamer, a ribozyme, a guide sequences for a ribozyme that inhibit translation or transcription of essential tumor proteins and genes, a hormone, an immunomodulator, an antipyretic, an anxiolytic, an antipsychotic, an analgesic, an antispasmodic, an anti-inflammatory agent, an anti-histamine, an anti-infective, a chemotherapeutic, or a combination thereof.

4. The decellularized tissue hydrogel of claim 1, wherein the decellularized tissue is decellularized brain, spinal cord, heart, lung, liver, muscle, cartilage, tendons, ligament, menisci, stomach, intestine, pancreas, kidney, blood vessel, bone, or cornea tissue.

5. The decellularized tissue hydrogel of claim 1, wherein the decellularized tissue hydrogel is formed by the method comprising:
(a) obtaining an amount of tissue from a donor;
(b) inducing widespread apoptosis in the tissue;
(c) removing apoptotic bodies using a mild hyper or hypo tonic buffered solution;
(d) removing DNA by exposing the tissue from (c) to DNase;
(e) enzymatically digesting the tissue from (d)
(f) neutralizing the tissue from (e) to form a pre-gel solution; and
(g) incubating the pre-gel solution at about 37° C. for an amount of time to form the decellularized tissue hydrogel.

6. The decellularized tissue hydrogel of claim 5, wherein step (b) is performed by incubating the tissue in (a) to camptothecin.

7. The decellularized tissue hydrogel of claim 5, wherein step (e) is performed by incubating the tissue from (d) in a solution comprising pepsin.

8. The decellularized tissue hydrogel of claim 5, wherein step (g) is performed in vitro.

9. The decellularized tissue hydrogel of claim 5, wherein step (g) is performed in vivo.

10. The decellularized tissue hydrogel of claim 5, further comprising the step of lyophilizing the tissue from step (d) before step (e).

11. The decellularized tissue hydrogel of claim 1, wherein the decellularized tissue hydrogel is formed by the method comprising:
(a) obtaining an amount of tissue from a donor;
(b) incubating the tissue from (a) in a solution containing sodium deoxycholate;
(c) removing DNA from the tissue from (b) by exposing the tissue from (b) to DNase;
(d) washing the tissue from (c) in PBS;
(e) enzymatically digesting the tissue from (d)
(f) neutralizing the tissue from (e) to form a pre-gel solution; and
(g) incubating the pre-gel solution at about 37° C. for an amount of time to form the decellularized tissue hydrogel.

12. The decellularized tissue hydrogel of claim 11, wherein step (b) is performed by incubating the tissue in about a 3% solution (w/w), (w/v), or (v/v) of sodium deoxycholate.

13. The decellularized tissue hydrogel of claim 11, wherein step (e) is performed by incubating the tissue from (d) in a solution comprising pepsin.

14. The decellularized tissue hydrogel of claim 11, wherein step (g) is performed in vitro.

15. The decellularized tissue hydrogel of claim 11, wherein step (g) is performed in vivo.

16. The decellularized tissue hydrogel of claim 11, further comprising the step of lyophilizing the tissue from step (d) before step (e).

17. A method comprising:
administering a decellularized tissue hydrogel of claim 1 to a subject.

18. The method of claim 17, further comprising co-administering a population of cells with the decellularized tissue hydrogel.

19. The method of claim 17, wherein the subject has a peripheral nerve injury, spinal cord injury, spinal cord disease, amputation, has degradation of a component of the spine, osteoarthritis, lung trauma, volumetric muscle loss, or liver cirrhosis.

20. A pre-gel solution comprising:
decellularized tissue, wherein the decellularized tissue contains native extracellular matrix proteins,
wherein the decellularized tissue is capable of forming a hydrogel at about 37° C.

21-35. (canceled)
